(19) United States
(12) Patent Application Publication
(10) Pub. No.: US 2026/0325542 A1
(43) Pub. Date: Oct. 22, 2026

(54) CANCER TARGETING COMPLEX, AND COMPOSITION COMPRISING SAME FOR PREVENTING OR TREATING CANCER

(71) Applicant: EWHA UNIVERSITY - INDUSTRY COLLABORATION FOUNDATION, Seoul (KR)

(72) Inventors: Kwangmeyung KIM, Seoul (KR); Seong lk JEON, Seoul (KR); Hanhee CHO, Seoul (KR)

(73) Assignee: EWHA UNIVERSITY - INDUSTRY COLLABORATION FOUNDATION, Seoul (KR)

(21) Appl. No.: 19/483,786

(22) PCT Filed: Dec. 15, 2023
(86) PCT No.: PCT/KR2023/020713

§ 371 Date: Nov. 12, 2025

(30) Foreign Application Priority Data
May 16, 2023 (KR) 10-2023-0063234

(51) Int. Cl. A61K 31/551; A61K 9/00; A61P 35/00

(52) U.S. Cl. CPC A61K 31/551; A61K 9/0019; A61P 35/00

(57) ABSTRACT

When administered in vivo, the complex for cancer targeting according to the present invention immediately binds to albumin and exists in a stable and inactivated state. Therefore, it can provide enhanced cancer treatment, prevention, and improvement effects specific to cancer tissue while exhibiting no toxicity to normal tissue.

### 1. FIELD

The present invention relates to a complex for cancer targeting, and more specifically, to a complex that targets cancer, is efficiently absorbed into cancer cells, and can effectively prevent or treat cancer, and a composition for preventing, treating, or improving cancer comprising the same.

### 2. DESCRIPTION OF THE RELATED ART

According to statistics from the International Agency for Research on Cancer (IARC) under the World Health Organization, the incidence of colorectal cancer in Korea ranked first in the world, and it is among the top cancers occurring in Koreans due to population aging, obesity, and changes in dietary habits. Various chemical therapeutic agents, immunotherapies, and targeted therapeutic agents have been developed for cancer treatment, and among them, targeted therapeutic agents are known to exhibit excellent effects in suppressing cell growth or inducing apoptosis by specifically targeting cancer cells. However, targeted therapeutic agents have a problem in that the therapeutic response varies greatly depending on the patient's genetic characteristics. In fact, anti-EGFR agents such as Cetuximab (Erbitux®) are known as effective first-line treatments for cancer. However, only a few patients are responsive to anti-EGFR therapies. Furthermore, anti-EGFR treatment involves significant costs, but it is difficult to predict drug responsiveness, resulting in anti-EGFR agents being administered to many patients in clinical practice, but the actual effect appears in only a minority of patients (20%).

To solve these problems, PROTACs (PROTAC) have been developed. Since they do not require access to the activation site of POIs, they were effective in treating proteins that are ‘hard to develop (undruggable)’. However, due to their small molecular structure and hydrophobicity, PROTACs have too low efficacy as anticancer agents, failing to achieve sufficient effects in clinical practice. Nanomaterials including micelles, liposomes, and nanoparticles have been developed to improve the shortcomings of PROTAC, but side effect problems caused by the nanomaterials used as carriers have been confirmed.

Therefore, there is an urgent need for the development of new platform-type anticancer agents that can solve the problems of PROTAC while achieving excellent anticancer effects.

### REFERENCES

1. Korean Registered Patent Publication No. 10-1930399

### SUMMARY

The present invention has been devised to solve the above problems, and the present invention intends to provide a complex for cancer targeting that exists in a stabilized and inactivated state by binding to albumin in vivo, and uses thereof.

Another objective of the present invention is to provide a pharmaceutical composition for preventing or treating cancer that can specifically penetrate cancer cells.

Another objective of the present invention is to provide a food composition for preventing or improving cancer as described above.

To achieve the above objectives, the present invention provides a complex for cancer targeting comprising: (i) a maleimide functional group; (ii) a linker cleavable by an esterase connected to the functional group; and (iii) a PROTAC connected to the linker.

The linker is a peptide linker comprising one or more amino acid sequences selected from the group consisting of G, GG, and GGG, a non-peptide linker selected from the group consisting of PABC (p-aminocarbamate), MBA (N,N′-methylenebisacrylamide), alkylene linker, arylene linker, polyalkylene oxide linker (e.g., PEG), NHS ester linker, Triazene linker, hydrazone linker, disulfide linker, glucuronide-MABC linker, Y—S—Z linker, and dialkoxydiphenylsilane linker, or a linker in which the peptide linker and the non-peptide linker are hybridized. The Y is azide (N₃), alkyl azide having 1 to 6 carbon atoms (—R′—N═N═N), the Z is any one selected from tetrazine, trans-cyclooctene, and cyclooctene, and the S is any one selected from oligo or poly(ethylene glycol) (PEG), dextran, and alkyl having 1 to 10 carbon atoms.

The PROTAC may target any one or more proteins selected from the group consisting of CDK9 (Cyclin-dependent kinase 9)/CDK family, BRD4 (Bromodomain-containing protein 4)/BET (Bromodomain and extra-terminal) family, SHP-2 (Src homology region 2 domain-containing phosphatase-2), AR (Androgen receptor), ER (Estrogen receptor), RAR (Retinoic acid receptor), ERRα (Estrogen-related receptor alpha), BTK (Bruton's tyrosine kinase), ALK (Anaplastic lymphoma kinase), RIPK (Receptor-interacting serine/threonine-protein kinase), c-Met (MET proto-oncogene/receptor tyrosine kinase), FAK (Focal adhesion kinase), IRAK4 (Interleukin-1 receptor-associated kinase 4), p38 MAPK (p38 mitogen-activated protein kinases), SGK (Serum and glucocorticoid-regulated kinase), TBK1 (TANK-binding kinase 1), KRAS (Kirsten rat sarcoma virus protein), B-Raf (B-Raf proto-oncogene serine/threonine kinase), β-catenin, FKBP (FK506 binding protein), IDO (Indoleamine 2,3-dioxygenase), PD-1/PD-L1 (Programmed death protein 1/programmed death-ligand 1), PARP1 (Poly [ADP-ribose] polymerase 1), PRC2 (Polycomb repressive complex 2), EGFR (Epidermal growth factor receptor), Sirt2 (NAD-dependent deacetylase sirtuin 2), HER2/ERBB2 (Human epidermal growth factor receptor 2), FRS2 (Fibroblast growth factor receptor substrate 2), BCL-XL (B-cell lymphoma-extra large), SMARCA (SWI/SNF related matrix associated actin dependent regulator of chromatin subfamily A), HDM2 (Human double minute 2 homolog), HDAC (Histone deacetylase) family, BCR-ABL (Breakpoint cluster region protein-Tyrosine-protein kinase ABL1), MCL1 (Modulator Of VRAC Current 1), FLT-3 (Fms Related Receptor Tyrosine Kinase 3), STAT3 (Transcription factor STAT3), Myc family, and BAF complex (Brg/Brahma-associated factors).

The linker may be Y—S—Z (where Y is an azide (N₃) group or alkyl azide having 1 to 6 carbon atoms (—R′—N═N═N, where R′ is an alkyl group having 1 to 6 carbon atoms), Z is trans-cyclooctene or cyclooctene, and S is poly(ethylene glycol)), or a hybridized linker of a peptide linker consisting of 1 to 3 glycines (G) and a poly(ethylene glycol) non-peptide linker.

The anticancer agent for cancer targeting may be represented by the following Chemical Formula 1 or 2.

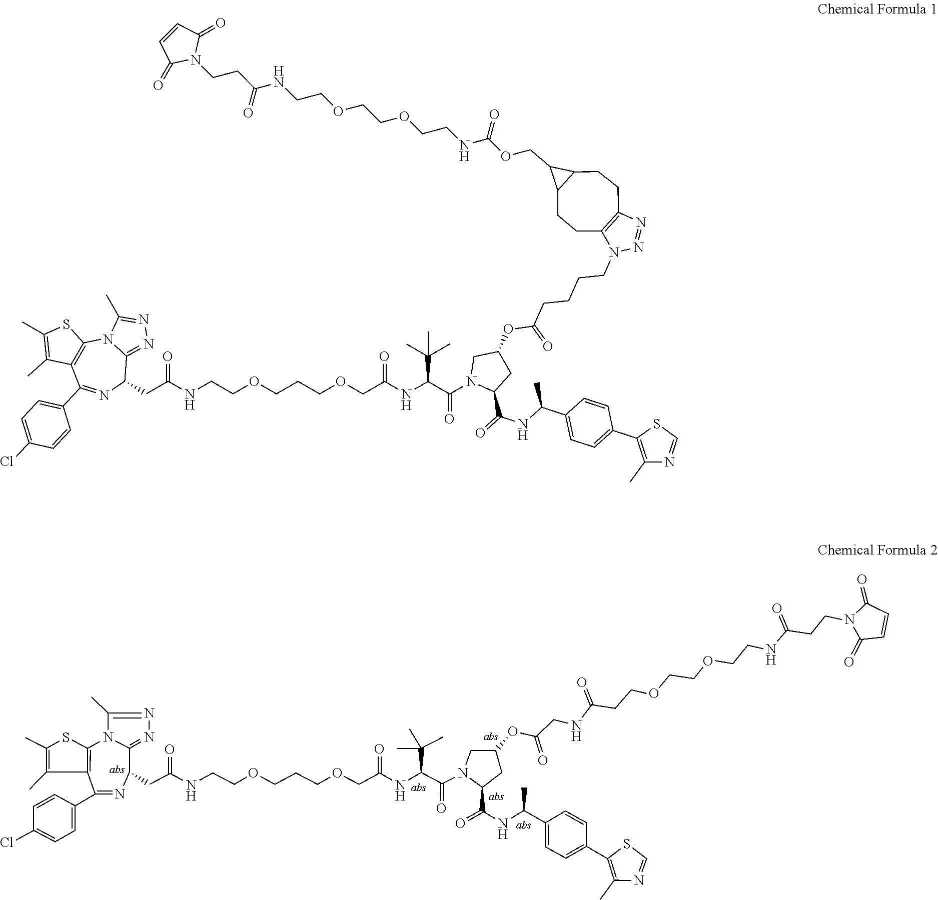

The complex for cancer targeting may be formed into a stabilized and inactivated form by binding to albumin present in vivo.

The complex for cancer targeting may penetrate into cancer cells and be cleaved by esterase present in the cancer cells to release PROTAC.

To achieve the other objective, the present invention provides a pharmaceutical composition for preventing or treating cancer comprising the complex for cancer targeting as an active ingredient.

The complex for cancer targeting may be formed into a stabilized and inactivated form by binding to albumin present in vivo.

The cancer may be any one or more selected from brain tumor, head and neck cancer, breast cancer, lung cancer, esophageal cancer, stomach cancer, duodenal cancer, appendiceal cancer, colorectal cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, and skin cancer.

To achieve the other objective, the present invention provides a food composition for preventing or improving cancer comprising the complex for cancer targeting as an active ingredient.

Furthermore, the present invention provides a method for preventing or treating cancer, comprising the step of administering the complex for cancer targeting to a subject.

Furthermore, the present invention provides the use of the complex for cancer targeting for the manufacture of a medicament for the prevention or treatment of cancer.

When administered in vivo, the complex for cancer targeting according to the present invention immediately binds to albumin and exists in a stable and inactivated state. Therefore, it can provide enhanced cancer treatment, prevention, and improvement effects specific to cancer tissue while exhibiting no toxicity to normal tissue.

In particular, the complex for cancer targeting according to the present invention penetrates deeply into not only cancer tissue located near blood vessels but also cancer cells located in the center of the cancer tissue. Therefore, the anticancer effect is significantly higher compared to the same dose of anticancer agent.

### BRIEF DESCRIPTION OF DRAWINGS

FIGS. 1A to 1E are diagrams schematically illustrating the structure of the complex for cancer targeting according to the present invention and its pharmacokinetic process. FIG. 1A shows the synthesis process of albumin-binding esterase cleavable BRD4-degrading PROTAC (ECMal-PROTAC) according to the present invention. FIG. 1B shows the process in which ECMal-PROTAC according to the present invention specifically binds and degrades through thiol-maleimated reaction with plasma albumin in the blood vessel. FIG. 1C shows the process in which ECMal-PROTAC bound to albumin is selectively accumulated in cancer tissue through the EPR effect. FIG. 1D shows the process in which albumin-bound ECMal-PROTAC is easily introduced selectively into cancer cells and cleaved inside the cancer cells to release free ARV-771. FIG. 1E shows the mechanism by which PROTAC acts within cancer cells, where free ARV-771 simultaneously binds to both BRD4 and VHL E3 ligase, inducing the polyubiquitination of BRD4. Subsequently, the BRD4/ARV-771/E3 ligase complex dissociates, and polyubiquitinated BRD4 is recognized by the proteasome and degraded into oligopeptides. The dissociated ARV-771 is recycled and participates in the degradation of other BRD4.

FIG. 1F shows the synthesis process of the complex for cancer targeting according to the present invention and the pharmacological mechanism in cancer cells.

FIG. 2 shows the chemical structure of the complex for cancer targeting (ECMal-PROTAC) according to the present invention.

FIG. 3A is the HPLC spectrum of ARV-771, N₃-PROTAC, and the complex for cancer targeting (ECMal-PROTAC).

FIG. 3B is the LC-MS spectrum of the complex for cancer targeting (ECMal-PROTAC) prepared from example 1 (m/z=1586.7, [M]+H⁺).

FIG. 3C and FIG. 3D are the HPLC and LC-MS spectra of ECMal-PROTAC prepared from example 2.

FIG. 4 is the HPLC spectrum of a mixture of BSA and the complex for cancer targeting (ECMal-PROTAC).

FIG. 5 is the LC-MS spectrum of a mixture of BSA (Alb) and the complex for cancer targeting (ECMal-PROTAC).

FIG. 6 is the HPLC spectrum for a mixture of BSA and ARV-771, a mixture of thiol-blocked BSA (thiol-blocked BSA) and the complex for cancer targeting (ECMal-PROTAC), the complex for cancer targeting (ECMal-PROTAC), and BSA.

FIG. 7 is a graph quantifying and showing the reduction in the HPLC peak area associated with the complex for cancer targeting (ECMal-PROTAC) from FIG. 4 and FIG. 6.

FIG. 8 is the result of DLS analysis for the complex for cancer targeting (ECMal-PROTAC) according to example 1 before and after binding to albumin (BSA, albumin).

FIG. 9 is the result of HPLC analysis over time after treating Albumin-ECMal-PROTAC with esterase (15 units).

FIG. 10 is a confocal microscope image of 4T1 cancer cells treated with Cy5.5-ARV-771 or Cy5.5-Alb-ECMal-PROTAC, and FIG. 11 is a graph quantifying and showing the intracellular fluorescence intensity in 4T1 cancer cells treated with Cy5.5-ARV-771 or Cy5.5-Alb-ECMal-PROTAC.

FIG. 12 is the result of Western blot analysis after treating 4T1 cancer cells with ARV-771 or the complex for cancer targeting (ECMal-PROTAC) of example 1.

FIGS. 13A and 13B are graphs quantifying the relative expression levels of c-Myc (FIG. 13A) and Bcl-2 (FIG. 13B) in 4T1 cancer cells treated with ARV-771 or the complex for cancer targeting (ECMal-PROTAC) of example 1.

FIG. 14 is a graph quantifying the BRD4 expression level according to the concentration of ARV-771 or the complex for cancer targeting (ECMal-PROTAC) of example 1.

FIGS. 15A and 15B are graphs analyzing the survival rate after treating 4T1 cancer cells or MDA-MB231 with the complex for cancer targeting (ECMal-PROTAC) of example 1 (FIG. 15A) and the complex for cancer targeting (Mal-PROTAC) of example 2 (FIG. 15B).

FIG. 16 is a pharmacokinetic profile analyzing the plasma concentration over time for the comparison group (Cy5.5-ARV-771 intravenous injection) and the example group (Cy5.5-Alb-ECMal-PROTAC intravenous injection).

FIG. 17 is an NIRF (non-invasive near-infrared fluorescence) image over time for the comparison group (Cy5.5-ARV-771 intravenous injection) and the example group (Cy5.5-Alb-ECMal-PROTAC intravenous injection).

FIG. 18 is a graph quantifying only the fluorescence signal in the cancer tissue from the results of the comparison group (Cy5.5-ARV-771 intravenous injection) and the example group (Cy5.5-Alb-ECMal-PROTAC intravenous injection) in FIG. 17 (****p<0.0001).

FIGS. 19A and 19B are the ex vivo fluorescence images (FIG. 19A) and quantified fluorescence intensity graph (FIG. 19B) of major organs (liver, lung, spleen, kidney, heart) and cancer tissue harvested from the comparison group (Cy5.5-ARV-771 intravenous injection) and the example group (Cy5.5-Alb-ECMal-PROTAC intravenous injection).

FIG. 20 is the fluorescence image (upper) and quantified fluorescence intensity graph (lower) of cancer tissue harvested from the comparison group (Cy5.5-ARV-771 intravenous injection) and the example group (Cy5.5-Alb-ECMal-PROTAC intravenous injection) (***p<0.001).

FIG. 21 is an enlarged fluorescence image of cancer tissue excised from the comparison group (Cy5.5-ARV-771 intravenous injection) and the example group (Cy5.5-Alb-ECMal-PROTAC intravenous injection).

FIG. 22 is an image taken with a confocal laser scanning microscope after staining cancer tissue excised from the comparison group (Cy5.5-ARV-771 intravenous injection) and the example group (Cy5.5-Alb-ECMal-PROTAC intravenous injection) with DAPI and the CD31 marker.

FIG. 23 is an image taken with a confocal laser scanning microscope after staining cancer tissue of the control group (Saline, control) administered with saline instead of the sample, with DAPI and the CD31 marker.

FIG. 24 is a graph diagramming the fluorescence signal on the cross-section of cancer tissue excised from the control group (control, Saline), the comparison group (Cy5.5-ARV-771 intravenous injection), and the example group (Cy5.5-Alb-ECMal-PROTAC intravenous injection).

FIG. 25 is a graph measuring and showing the cancer size measured from the control group, the comparison group, and the example group over time (ns is indicated if no significant difference was shown. ****p<0.0001).

FIG. 26 is an optical image of cancer tissue excised from the control group, the comparison group, and the example group.

FIG. 27 is a confocal laser scanning image taken after TUNEL staining of cancer tissue excised from the control group, the comparison group, and the example group.

FIG. 28 is a graph analyzing and showing the weight change of the control group, the comparison group, and the example group.

FIG. 29 is the result of histological analysis on organ tissue (liver, lung, spleen, kidney, heart) and cancer tissue excised from the control group, the comparison group, and the example group.

FIG. 30 is the result of TUNEL analysis on liver tissue excised from the control group, the comparison group, and the example group.

FIG. 31 is the result of Western blot analysis of the expression levels of BRD4, c-Myc, and Bcl-2 in cancer tissue excised from the control group, the comparison group, and the example group.

FIG. 32 is a graph quantifying and showing the results of FIG. 31 (**p<0.01, ****p<0.0001).

FIG. 33 is an image taken with a confocal laser scanning microscope after staining cancer tissue excised from the control group (Control, Saline), the comparison group (ARV-771, intravenous injection), and the example group (ECMal-PROTAC, intravenous injection) with DAPI, anti-BRD4, and anti-c-Myc.

FIG. 34 is the result of DAB staining of cancer tissue excised from the control group, the comparison group, and the example group.

### DETAILED DESCRIPTION

Hereinafter, the present invention will be described in detail.

Unless defined otherwise, all technical and scientific terms used herein have the same meaning as commonly understood by one of ordinary skill in the art to which the present invention belongs. Generally, the nomenclature used herein is well known and commonly used in the art.

In this specification, when a part is referred to as “comprising” a certain component, it means that it may further include other components, rather than excluding other components, unless there is a specific description to the contrary.

One aspect of the present invention relates to a complex for cancer targeting, comprising: (i) a maleimide functional group; (ii) a linker cleavable by an esterase connected to the functional group; and (iii) a PROTAC connected to the linker.

The linker is a peptide linker comprising one or more amino acid sequences selected from the group consisting of G, GG, and GGG, a non-peptide linker selected from the group consisting of PABC (p-aminocarbamate), MBA (N,N′-me-thylenebisacrylamide), alkylene linker, arylene linker, polyalkylene oxide linker (e.g., PEG), NHS ester linker, Triazene linker, hydrazone linker, disulfide linker, glucuronide-MABC linker, Y—S—Z linker, and dialkoxydiphenylsilane linker, or a linker in which the peptide linker and the non-peptide linker are hybridized. The Y is azide (N₃), alkyl azide having 1 to 6 carbon atoms (—R′—N═N═N), the Z is any one selected from tetrazine, trans-cyclooctene, and cyclooctene, and the S may be any one selected from oligo or poly(ethylene glycol) (PEG), dextran, and alkyl having 1 to 10 carbon atoms.

When administered in vivo, the complex for cancer targeting according to the present invention binds to endogenous albumin present in the blood vessel in vivo through a thiol-maleimide reaction. Due to this structural feature, the complex for cancer targeting according to the present invention not only improves the problems of low bioavailability, low targeting, and accumulation rate in tumor tissue that conventional PROTACs had, but also has significantly excellent effects in cancer treatment, prevention, and improvement.

The complex for cancer targeting according to the present invention exists as an albumin-bound complex, is easily accumulated inside cancer cells through endocytosis in cancer cells, releases PROTAC by esterase present inside the cancer cells, and restores BRD4 degradation activity. Thereby, it can have a cancer cell-specific apoptotic effect.

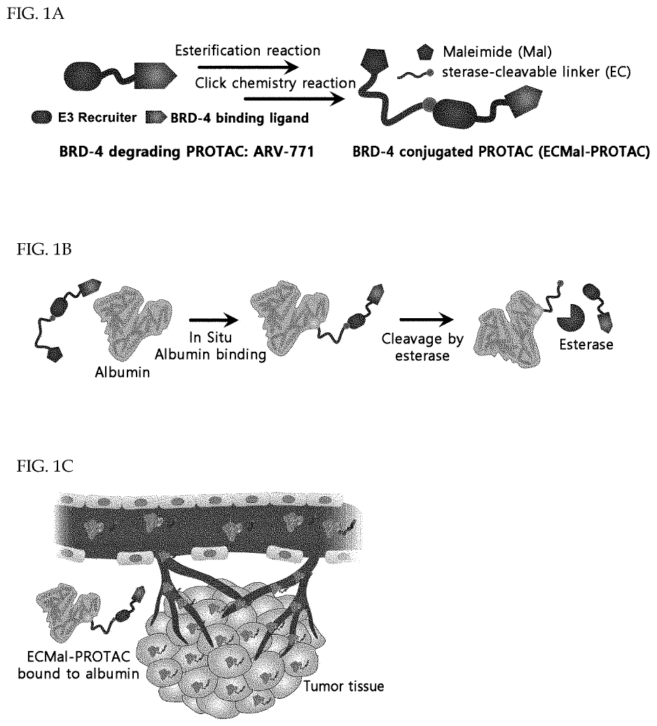

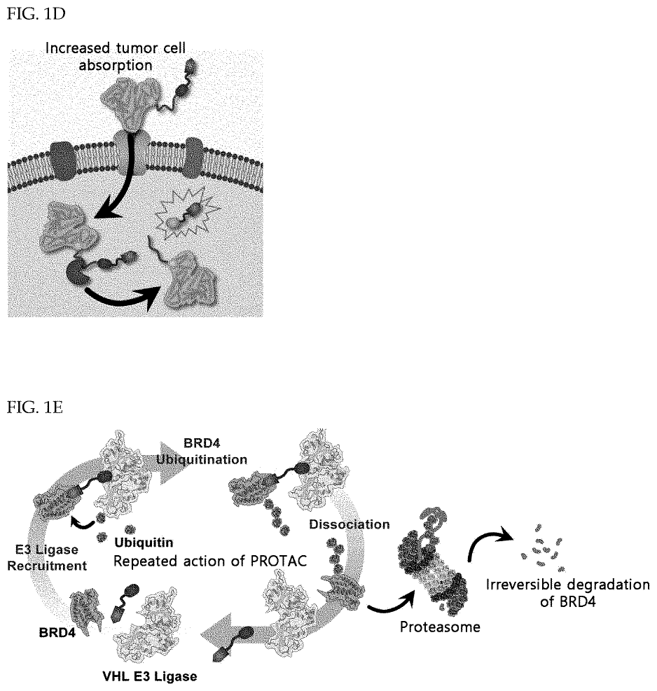

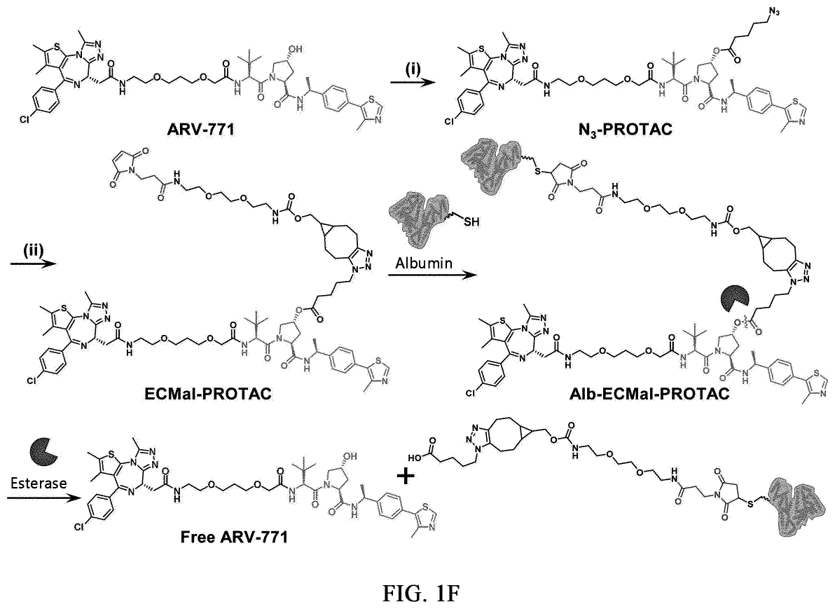

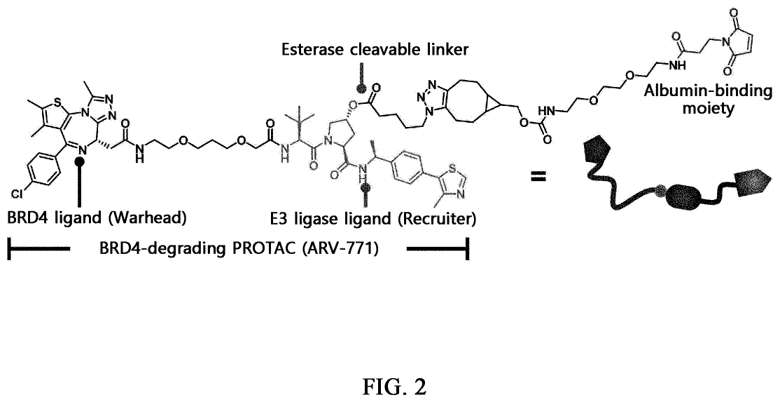

FIGS. 1A to 1E schematically illustrate the structure and pharmacokinetic mechanism of the complex for cancer targeting (ECMal-PROTAC) according to the present invention. Referring to this, the complex for cancer targeting of the present invention is a complex formed by the binding of a maleimide functional group (ECMal) capable of binding to albumin, with a PROTAC, and the maleimide functional group and the PROTAC may be directly bound or bound through a linker.

The usable linker is not particularly limited as long as it is a linker cleavable specifically by esterase. However, preferably, it is a peptide linker comprising one or more amino acid sequences selected from the group consisting of G, GG, and GGG, a non-peptide linker selected from the group consisting of PABC (p-aminocarbamate), MBA (N,N′-methylenebisacrylamide), alkylene linker, arylene linker, polyalkylene oxide linker (e.g., PEG), NHS ester linker, Triazene linker, hydrazone linker, disulfide linker, glucuronide-MABC linker, Y—S—Z linker, and dialkoxydiphenylsilane linker, or a linker in which the peptide linker and the non-peptide linker are hybridized. The Y is azide (N₃), alkyl azide having 1 to 6 carbon atoms (—R′—N═N═N), the Z is any one selected from tetrazine, trans-cyclooctene, and cyclooctene, and the S may be any one selected from oligo or poly(ethylene glycol) (PEG), dextran, and alkyl having 1 to 10 carbon atoms.

In the present invention, the term “peptide linker” refers to a linker consisting only of peptide residues. It is not particularly limited as long as it is cleavable by the esterase enzyme specifically existing in excess inside cancer cells. Preferably, it may be any one or more selected from G, GG, and GGG.

In the present invention, the term “non-peptide linker” refers to a linker composed of a compound that is not a peptide. It is not particularly limited as long as it is cleavable by the esterase enzyme specifically existing in excess inside cancer cells. However, preferably, it may be any one or more selected from the group consisting of PABC (p-aminocarbamate), MBA (N,N′-methylenebisacrylamide), alkylene linker, arylene linker, polyalkylene oxide linker (e.g., PEG), NHS ester linker, Merrifield linker, Wang linker, Sasrin linker, Tritiyl linker, RINK-amide linker, Kenner linker, Silyl linker, Triazene linker, hydrazone linker, disulfide linker, glucuronide-MABC linker, Y—S—Z linker, and dialkoxydiphenylsilane linker. The Y is azide (N₃), alkyl azide having 1 to 6 carbon atoms (—R′—N═N═N), the Z is any one selected from tetrazine, trans-cyclooctene, and cyclooctene, and the S may be any one selected from oligo or poly(ethylene glycol) (PEG), dextran, and alkyl having 1 to 10 carbon atoms. Preferably, it may be a polyalkylene oxide linker (e.g., PEG) or a Y—S—Z linker.

In the present invention, the alkylene linker may be an alkylene linker having 2 to 20 carbon atoms, and preferably an alkylene linker having 2 to 8 carbon atoms. The arylene linker may be a linker consisting of an arylene group having 6 to 30 carbon atoms.

The alkyl azide is an alkyl azide having 1 to 6 carbon atoms, and may be represented by —R′—N═N═N, where R′ may be an alkyl group having 1 to 6 carbon atoms.

The cyclooctene may be any one selected from the group consisting of dibenzylcyclooctyne (DBCO), difluorocyclooctyne (DIFO), bicyclononyne (BCN), dibenzoazacyclooctyne (DIBAC), dibenzocyclooctynol (DIBO), and azadibenzocyclooctyne (ADIBO).

In the present invention, the term “linker in which the peptide linker and the non-peptide linker are hybridized” means a linker formed by the binding of a peptide linker and a non-peptide linker. The binding order is not particularly limited, but preferably, it may be one bound in the order of peptide linker-non-peptide linker. A preferred example of the hybridized linker of the peptide linker and the non-peptide linker may be G-(PEG)ₘ (m=1-5), GG-(PEG)ₘ (m=1-5), or GGG-(PEG)ₘ (m=1-5).

In one embodiment of the present invention, the linker (ii) may be Y—S—Z (where Y is an azide (N₃) group or alkyl azide having 1 to 6 carbon atoms (—R′—N═N═N, where R′ is an alkyl group having 1 to 6 carbon atoms), Z is trans-cyclooctene or cyclooctene, and S is poly(ethylene glycol)), or a hybridized linker of a peptide linker consisting of 1 to 3 glycines (G) and a poly(ethylene glycol) non-peptide linker.

The complex for cancer targeting of the present invention is cleaved at the bond between the PROTAC and the linker by esterase present inside the cancer cells, thereby releasing PROTAC. At this time, if even a small part of the linker compound remains at one end of the PROTAC, or if loss or damage occurs to the structure of the PROTAC, a problem arises where the activity is greatly reduced. Considering these points, the present inventors performed experiments on various linkers and confirmed that it is preferable to use a linker cleavable by esterase, thereby completing the complex for cancer targeting of the present invention.

The linker used in the present invention has the advantage that it can completely cleave without damage or residue of the PROTAC by esterase present in cancer cells, thus maintaining the efficacy of the PROTAC as it is.

The term “proteolysis-targeting chimera (PROTAC)” used in the present invention is a platform technology composed of an E3 ligase binding module-conjugate target protein binding module. It can induce in vivo degradation through ubiquitination of the disease-causing target protein. It can inhibit most disease-causing proteins that are difficult to inhibit with active site-directed drugs derived from conventional drug development methods, by degrading them.

The PROTAC may target any one or more proteins selected from the group consisting of CDK9 (Cyclin-dependent kinase 9)/CDK family, BRD4 (Bromodomain-containing protein 4)/BET (Bromodomain and extra-terminal) family, SHP-2 (Src homology region 2 domain-containing phosphatase-2), AR (Androgen receptor), ER (Estrogen receptor), RAR (Retinoic acid receptor), ERRα (Estrogen-related receptor alpha), BTK (Brfvuton's tyrosine kinase), ALK (Anaplastic lymphoma kinase), RIPK (Receptor-interacting serine/threonine-protein kinase), c-Met (MET proto-oncogene/receptor tyrosine kinase), FAK (Focal adhesion kinase), IRAK4 (Interleukin-1 receptor-associated kinase 4), p38 MAPK (p38 mitogen-activated protein kinases), SGK (Serum and glucocorticoid-regulated kinase), TBK1 (TANK-binding kinase 1), KRAS (Kirsten rat sarcoma virus protein), B-Raf (B-Raf proto-oncogene serine/threonine kinase), β-catenin, FKBP (FK506 binding protein), IDO (Indoleamine 2,3-dioxygenase), PD-1/PD-L1 (Programmed death protein 1/programmed death-ligand 1), PARP1 (Poly [ADP-ribose]polymerase 1), PRC2 (Polycomb repressive complex 2), EGFR (Epidermal growth factor receptor), Sirt2 (NAD-dependent deacetylase sirtuin 2), HER2/ERBB2 (Human epidermal growth factor receptor 2), FRS2 (Fibroblast growth factor receptor substrate 2), BCL-XL (B-cell lymphoma-extra large), SMARCA (SWI/SNF related matrix associated actin dependent regulator of chromatin subfamily A), HDM2 (Human double minute 2 homolog), HDAC (Histone deacetylase) family, BCR-ABL (Breakpoint cluster region protein-Tyrosine-protein kinase ABL1), MCL1 (Modulator Of VRAC Current 1), FLT-3 (Fms Related Receptor Tyrosine Kinase 3), STAT3 (Transcription factor STAT3), Myc family, and BAF complex (Brg/Brahma-associated factors).

The E3 ligase that the PROTAC operates on may exhibit a protein degradation effect through an E3 ligase selected from the group consisting of CRBN (Cereblon), VHL (Von Hippel-Lindau tumor suppressor), cIAP (Cellular inhibitor of apoptosis protein), Keap1 (Kelch-like ECHassociated protein 1), HDM2 (Human double minute 2 homolog), βTRCP (Beta-transducin repeat containing E3 ubiquitin protein ligase), CHIP (Carboxyl terminus of HSC70-interacting protein), and UHRF1 (ubiquitin like with PHD and ring finger domains 1). Specifically, it may exhibit a protein degradation effect through an E3 ligase selected from the group consisting of CRBN (Cereblon) and VHL (Von Hippel-Lindau tumor suppressor).

In one embodiment of the present invention, the complex for cancer targeting according to the present invention may be represented by the following Chemical Formula 1 or 2.

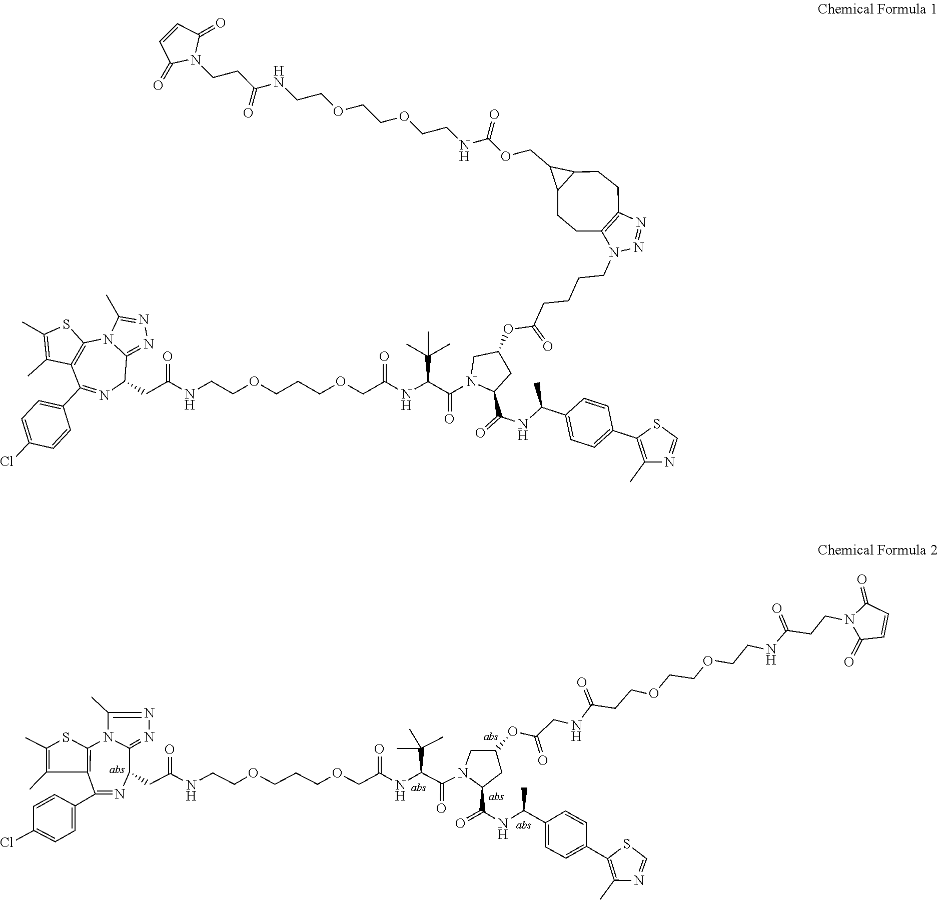

The complex for cancer targeting binds to albumin present in vivo and is formed into a stabilized and inactivated form. Therefore, even if present in large amounts in vivo, it does not exhibit toxicity in normal cells, which are not cancer tissue.

The complex for cancer targeting penetrates into cancer cells, is cleaved by esterase present in the cancer cells to release PROTAC, and thus has an excellent anticancer effect.

The molecular weight of the complex for cancer targeting may be 15 kDa to 25 kDa. If it exceeds the molecular weight, aggregation occurs, making it impossible to effectively enter cancer cells. If it is less than the molecular weight, there is a problem that it is easily eliminated through the kidneys, reducing the retention time in the body.

The complex for cancer targeting according to the present invention is characterized by being cleaved by esterase. Esterase is a suitable target substance for the structural characteristics of the present invention because it is overexpressed in cancer cells even without external stimulation. Most conventional prodrug anticancer agents target caspase. If caspase is targeted due to the structural characteristics of the present invention, a problem may arise in that the release of PROTAC does not easily occur unless stimulation by an anticancer agent precedes the cancer cell, increasing the concentration of caspase.

In one embodiment of the present invention, the PROTAC may be ARV-771. ARV-771 is one of the BRD4-degrading PROTACs that is composed of a BRD4 (bromodomain 4) inhibitor (JQ1) and a ligand for VHL (Von Hippel-Lindau) E3 ligase, and treats cancer by degrading BET (bromodomain and extra-terminal domain) protein and bromodomain. BET protein is known to be overexpressed in cancer cells. The complex for cancer targeting according to the present invention is bound to the PROTAC through a linker connected to the maleimide group that forms a bond with albumin. Due to this structural feature, when the complex for cancer targeting according to the present invention is administered in vivo, it forms a bond with albumin present in vivo, allowing for excellent tumor-specific accumulation and cellular absorption rate.

The synthesis process of the complex for cancer targeting according to the present invention and its pharmacological mechanism on cancer cells are illustrated in FIGS. 1A to 1E. In one example of the present invention, a ARV-771 having an azide group (N₃-PROTAC) was prepared by conjugating PROTAC with 5-azidovaleric acid through Steglich esterification. Next, the complex for cancer targeting (ECMal-PROTAC) was synthesized through a copper-free alkyne-azide click reaction between N₃-PROTAC and BCN-PEG2-Mal. To increase the purity of the complex for cancer targeting prepared through the above process, a purification step may be further included. Preferably, column chromatography may be used.

Another aspect of the present invention relates to a pharmaceutical composition for preventing or treating cancer, comprising the complex for cancer targeting as an active ingredient.

The complex for cancer targeting binds to albumin present in vivo to form a stabilized and inactivated form. It specifically accumulates in cancer cells and is specifically degraded by esterase present in cancer cells, releasing PROTAC, thereby inducing apoptosis in cancer cells.

The term “cancer” as used in the present invention generally refers to diseases caused by cells having aggressive characteristics (dividing and growing ignoring normal growth limits), invasive characteristics (infiltrating surrounding tissue), and metastatic characteristics (spreading to other parts of the body).

In the present invention, the type of cancer is not particularly limited. It may be brain tumor, head and neck cancer, breast cancer, lung cancer, esophageal cancer, stomach cancer, duodenal cancer, appendiceal cancer, colorectal cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, or skin cancer. In one example of the present invention, an anticancer effect was confirmed by co-administration of IU1 series compounds and PROTAC in uterine cancer and prostate cancer cell lines, but it is not limited thereto.

The term “prevention” as used in the present invention means all actions that suppress the cancer or delay the onset by administration of the pharmaceutical composition according to the present invention.

The term “treatment” as used in the present invention means all actions that improve or beneficially change the symptoms caused by the cancer by administration of the pharmaceutical composition according to the present invention.

The present inventors confirmed through specific examples that the complex for cancer targeting according to the present invention can enhance the in vivo retention time, cancer cell influx rate, and anticancer effect of PROTAC.

In one example of the present invention, it was confirmed that the complex for cancer targeting reduces cancer proliferation by 5 to 6 times compared to the case where PROTAC is administered alone. It was also confirmed that it causes damage only to cancer cells through apoptosis without causing any damage to normal cells, thereby significantly enhancing the anticancer effect.

The present inventors confirmed through various experimental examples below that administering the complex for cancer targeting of the present invention can significantly enhance cancer-specific selectivity, absorption rate into cancer cells, wide distribution to cancer cells, degradation effect on the target protein of PROTAC, and anticancer effect, and thus can be widely utilized in the field of cancer treatment.

The “complex for cancer targeting,” etc., may be within the scope described above.

The term “administration” in the present invention refers to introducing the pharmaceutical composition of the present invention to a subject by any suitable method. The route of administration may be oral or parenteral through various routes as long as it can reach the target tissue. Examples of parenteral administration routes may include subcutaneous, intraperitoneal, intrapulmonary, intranasal, intramuscular, intravenous, or intraarterial. Preferably, it may be intravenous administration.

The pharmaceutical composition of the present invention may further comprise appropriate carriers, excipients, or diluents commonly used in the manufacture of pharmaceutical compositions. Compositions comprising pharmaceutically acceptable carriers may be in various oral or parenteral dosage forms. When formulated, they may be prepared using diluents or excipients commonly used, such as fillers, expanders, binders, wetting agents, disintegrants, and surfactants. Formulations for oral administration may include tablets, pills, powders, granules, capsules, suspensions, internal solutions, emulsions, syrups, sterilized aqueous solutions, non-aqueous solvents, freeze-dried preparations, and the like. These formulations may be prepared by mixing one or more compounds with at least one excipient, for example, starch, calcium carbonate, sucrose, or lactose, gelatin, and the like.

In addition, lubricants such as magnesium stearate, talc, and the like may be used in addition to simple excipients. Liquid formulations for oral administration include suspensions, internal solutions, emulsions, and syrups, and may include various excipients, such as wetting agents, sweeteners, fragrances, preservatives, and the like, in addition to water and liquid paraffin, which are commonly used simple diluents. Formulations for parenteral administration may include sterilized aqueous solutions, non-aqueous solvents, suspensions, emulsions, freeze-dried preparations, and suppositories. Propylene glycol, polyethylene glycol, and vegetable oils such as olive oil may be used as non-aqueous solvents and suspending agents. Witepsol, macrogol, Tween 61, cocoa butter, laurin butter, glycerogelatin, and the like may be used as bases for suppositories.

Furthermore, the pharmaceutical composition of the present invention is administered in a pharmaceutically effective amount. The term “pharmaceutically effective amount” as used in the present invention means an amount sufficient to suppress or alleviate cancer at a reasonable benefit/risk ratio applicable to medical use. The effective dosage level may be determined according to factors including the type and severity of the subject, age, weight, diet, gender, drug activity, sensitivity to the drug, time of administration, route of administration and excretion rate, duration of treatment, co-administered drugs, and other factors well known in the medical field.

The administration of the pharmaceutical composition of the present invention may be once a day, several times dividedly, once every other day, or once a week. Specifically, the pharmaceutical composition may be administered at 0.001 to 1000 mg/kg/day, and more specifically at 0.1 to 100 mg/kg/day.

Furthermore, the composition of the present invention may be administered as an individual therapeutic agent or in combination with other therapeutic agents. When administered in combination with other therapeutic agents, the composition of the present invention and the other therapeutic agent may be administered sequentially or simultaneously. The other therapeutic agent may be a drug such as a compound or protein that promotes cancer regression or further prevents cancer proliferation or growth. It also includes all other anticancer therapies besides drug therapy, such as radiation therapy.

Another aspect of the present invention relates to a food composition for preventing or improving cancer, comprising the complex for cancer targeting as an active ingredient.

The “complex for cancer targeting,” “PROTAC,” “cancer,” “prevention,” etc., may be within the scope described above.

The food composition of the present invention is excellent in preventing or improving cancer.

The term “improvement” may mean all actions that reduce at least a parameter related to the condition being treated, for example, the severity of the symptoms. In this case, the food composition may be used simultaneously with or separately from a medicament for treatment, either before the onset stage or after the onset of the disease, for the prevention or improvement of cancer.

In the food composition, the active ingredient may be added directly to the food or used together with other foods or food components. It may be appropriately used according to conventional methods. The mixing amount of the active ingredient may be suitably determined according to its purpose of use (for prevention or improvement). Generally, when manufacturing foods or beverages, the food composition may be added in an amount of specifically about 15 wt % or less, and more specifically about 10 wt % or less, relative to the raw material. However, in the case of long-term consumption for health and hygiene purposes or health control purposes, the amount may be less than the above range.

The food composition may be formulated into one selected from the group consisting of tablets, pills, powders, granules, dusts, capsules, and liquid dosage forms, further comprising one or more of carriers, diluents, excipients, and additives. Foods to which the compound according to one aspect can be added include various food types, powders, granules, tablets, capsules, syrups, beverages, gums, teas, vitamin complex preparations, health functional foods, and the like.

Specific examples of the carrier, excipient, diluent, and additive may be one or more selected from the group consisting of lactose, dextrose, sucrose, sorbitol, mannitol, erythritol, starch, acacia gum, calcium phosphate, alginate, gelatin, calcium phosphate, calcium silicate, microcrystalline cellulose, polyvinylpyrrolidone, cellulose, polyvinylpyrrolidone, methylcellulose, water, sugar syrup, methylcellulose, methylhydroxybenzoate, propylhydroxybenzoate, talc, magnesium stearate, and mineral oil.

The food composition may contain other ingredients as essential components without special limitations, in addition to containing the active ingredient. For example, it may contain various flavorings or natural carbohydrates as additional ingredients, like conventional beverages. Examples of the natural carbohydrates mentioned above may be conventional sugars such as monosaccharides, e.g., glucose, fructose, etc.; disaccharides, e.g., maltose, sucrose, etc.; and polysaccharides, e.g., dextrin, cyclodextrin, etc., and sugar alcohols such as xylitol, sorbitol, erythritol, and the like. As flavorings other than those mentioned above, natural flavorings (thaumatin, stevia extract (e.g., rebaudioside A, glycyrrhizin, etc.)) and synthetic flavorings (saccharin, aspartame, etc.) can be advantageously used. The ratio of the natural carbohydrates can be appropriately determined by selection by those skilled in the art.

In addition to the above, the food composition of the present invention may contain various nutrients, vitamins, minerals (electrolytes), flavorings such as synthetic flavorings and natural flavorings, coloring agents, enhancers (cheese, chocolate, etc.), pectic acid and its salts, alginic acid and its salts, organic acids, protective colloid thickeners, pH adjusting agents, stabilizers, preservatives, glycerin, alcohol, carbonating agents used in carbonated drinks, and the like. These components can be used independently or in combination, and the ratio of these additives can also be appropriately selected by those skilled in the art.

The food composition can be used as a health functional food for the prevention or improvement of cancer.

The food composition can be provided mixed with conventionally known health functional foods for the prevention or improvement of cancer or newly developed health functional foods for the prevention or improvement of cancer.

If the health functional food further comprises a health functional food for the prevention or improvement of cancer, it is important to mix an amount that can achieve the maximum effect with the minimum amount without side effects, and this can be easily determined by those skilled in the art.

Another aspect of the present invention provides a method for preventing or treating cancer, comprising the step of administering the complex for cancer targeting to a subject.

The “complex for cancer targeting,” “PROTAC,” “administration,” “cancer,” “prevention,” “treatment,” etc., may be within the scope described above.

The term “subject” in the present invention means all animals including humans who are likely to develop the cancer or have developed it. By administering the composition of the present invention to the subject, cancer can be alleviated or treated.

The method may be co-administered with a known composition having an effect of preventing or treating cancer or another pharmaceutical composition. It may be administered simultaneously, separately, or sequentially, and may be administered singly or multiply.

Considering all the above factors, it is important to administer an amount that can achieve the maximum effect with the minimum amount without side effects, and this can be easily determined by those skilled in the art.

Another aspect of the present invention provides the use of the complex for cancer targeting for the manufacture of a medicament for the prevention or treatment of cancer.

The “cancer,” “prevention,” “treatment,” “complex for cancer targeting,” “PROTAC,” etc., may be within the scope described above.

Hereinafter, the present invention will be described in more detail through examples, etc. However, the scope and content of the present invention cannot be reduced or limited by the examples below. Furthermore, based on the disclosure of the present invention including the examples below, it is obvious that a person skilled in the art can easily practice the present invention for which specific experimental results are not presented. It is also natural that such variations and modifications fall within the scope of the appended claims.

In addition, the experimental results presented below only describe representative experimental results of the examples and comparative examples. The effect of each of the various embodiments of the present invention not explicitly presented below will be specifically described in the relevant section.

### Experimental Materials

In this example, ARV-771 (99.02%) was obtained from Chemscene (Monmouth Junction, NJ, USA). DMF (N,N-dimethylformamide; 99.8%), EDC-HCl (N-(3-dimethylaminopropyl)-N′-ethylcarbodiimide hydrochloride), DMAP (4-dimethylaminopyridine; 99%), esterase obtained from porcine liver, and TEA (triethylamine; 99.5%) were purchased and used from Sigma-Aldrich (St. Louis, MO, USA). 5-Azidovaleric acid (98%), 4-maleimidobutyric acid (98%), and 2-mercaptoethanol (98%) were commercially obtained and used from Tokyo Chemical Industry (TCI; Toshima, Tokyo, Japan). BCN-PEG2-Mal (Bicyclononyne-polyethylene glycol-maleimide; 95%) was purchased from Axispharm (San Diego, CA, USA), and acetonitrile (ACN) was purchased from Honeywell (Charlotte, NC, USA). DMSO (Dimethyl sulfoxide; 99%), BSA (bovine serum albumin; 98%), and TWEEN 20 were purchased from Biosesang (Ansan, Gyeonggi, Republic of Korea). Cy5.5-NHS(Cyanine 5.5-N-hydroxysuccinimide ester) and Cy5.5-COOH (cyanine 5.5-carboxylic acid) were purchased from Lumiprobe (Hunt Valley, MD, USA). Anti-BRD4, anti-c-Myc, anti-Bcl-2, and anti-β-actin were purchased from Abcam (Discovery Drive, Cambridge, United Kingdom). CCK-8 (Cell counting kit-8) was purchased from Vitascientific (Beltsville, MD, USA). RPMI (Roswell Park Memorial Institute) 1640 medium, FBS (fetal bovine serum), penicillin, and streptomycin were purchased from WELGENE Inc (Daegu, Republic of Korea). 4T1 (mouse breast cancer cells) cell line was purchased from American Type Culture Collection (ATCC; Manassas, VA, USA). All reagents were used as received.

### Experimental Equipment

Reverse-phase high-performance liquid chromatography (RP-HPLC; 1200 series, Agilent Technologies, Palo Alto, CA, USA) equipped with a C18 column (Eclipse XDB-C18, 4.6×150 mm, particle size=5 μm, Agilent Technologies) was used. The molar mass and their m/z (mass-to-charge ratios) of the products in all synthesis procedures were confirmed by liquid chromatography-mass spectrometry (LC-MS; Q-TOF 5600, AB SCIEX, Framingham, MA, USA). The hydrodynamic size of albumin was measured through dynamic light scattering (DLS; Zetasizer Nano ZS, Malvern Instruments, Malvern, UK). The chemical conjugation of FITC or Cy 5.5 dye to ARV-771 or BSA was verified by a fluorescence spectrophotometer (FL; Hitachi F-7000, Hitachi High-Tech, Hitachinaka, Ibaraki, Japan). Observation was performed using a confocal laser scanning microscope (CLSM; Leica TCS SP8, Leica Microsystems GmbH, Wetzlar, Germany) and a microplate reader (VERSAmax™, Molecular Devices Corp., Sunnyvale, CA, USA). The IVIS Lumina Series III system (PerkinElmer, Waltham, MA, USA) was used together with Living Image software (PerkinElmer) to investigate the in vivo and ex vivo biodistribution of the complex for cancer targeting (ECMal-PROTAC).

### Statistical Analysis

All statistical data of the present invention are presented as mean±standard deviation (SD) from at least three independent experiments. Statistical analysis between two groups was performed using Student's t-test. One-way analysis of variance (ANOVA) test was used to compare two or more groups, and statistical significance for multiple comparisons was verified by Tukey-Kramer. The p-value of each statistical data is indicated numerically with an asterisk (*p<0.05, **p<0.01, ***p<0.001, ****p<0.0001).

### Preparation Example 1. Preparation of Cy5.5 Conjugated with BSA and ARV-771

Cy5.5-conjugated ARV-771 and BSA were synthesized to experiment with the cellular uptake in vitro, pharmacokinetic properties in vivo, and biodistribution of Alb-ECMal-PROTAC and ARV-771. Specifically, ARV-771 (5 mg, 5.07 μmol), Cy5.5-COOH (5.8 mg, 7.60 μmol), and DMAP (3.1 mg, 25.34 μmol) were placed in a 4 mL glass vial, and 1 mL of anhydrous DMF was added to dissolve them. The EDC-HCl (3.9 mg, 25.34 μmol) was added to the vial. This activates the carboxylic acid group of Cy5.5-COOH. The crude product was obtained by reacting at room temperature for 12 hours. The pure Cy5.5-conjugated ARV-771 (Cy5.5-ARV-771) was recovered by passing the crude product through a C18 column using ACN/H2O (40/60) as the eluent.

To obtain Cy5.5-labeled BSA (Cy5.5-Alb), unlabeled BSA (66.5 mg, 1.00 μmol) and Cy5.5-NHS (0.1 mg, 0.10 μmol) were mixed and dissolved in 10 mL of anhydrous DMF. The reaction mixture was obtained by reacting at room temperature for 12 hours. After the reaction was completed, the reaction mixture was recovered, diluted with H2O, dialyzed using a dialysis membrane (MWCO=10,000 Da), and lyophilized to prepare Cy5.5-Alb, which had a 1:10 molar ratio of Cy5.5 to BSA.

The fluorescence intensities of Cy5.5-ARV-771 and Cy5.5-Alb were quantified by FL. Cy5.5-ARV-771 was further blended with native ARV-771 so that the molar ratio of Cy5.5 and ARV-771 was 1:10.

### Example 1. Synthesis of ECMal-PROTAC

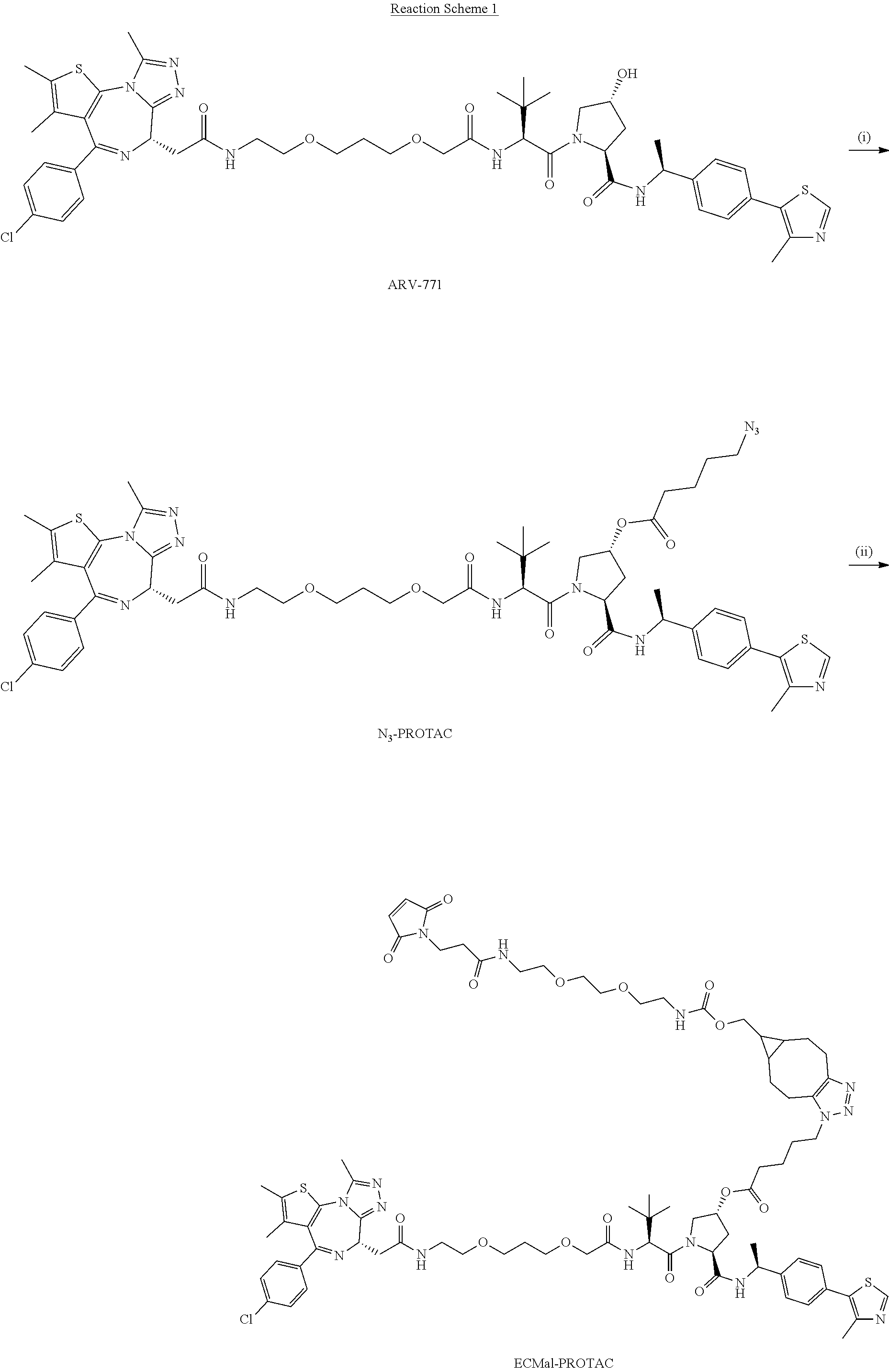

ARV-771 (20.0 mg, 20.27 μmol) was placed in a 3 ml 1-neck round-bottom flask equipped with a magnetic stirrer bar, and 0.5 ml of anhydrous DMF was added to dissolve it.

After successfully adding 5-azidovaleric acid (7.25 mg, 50.68 μmol), DMAP (12.38 mg, 101.35 μmol), and EDC-HCl (15.73 mg, 101.35 μmol) to the flask, the mixture was slowly mixed and reacted at room temperature for 12 hours. As a result, the crude product was concentrated under reduced pressure, and separated through C18 column chromatography (Sep-Pak C18 cartridges 5 g; Waters Corporation, Milford, MA, USA) using eluent of ACN/H2O (70/30, v/v) to obtain N₃-PROTAC. The recovered N₃-PROTAC was lyophilized to remove residual solvent.

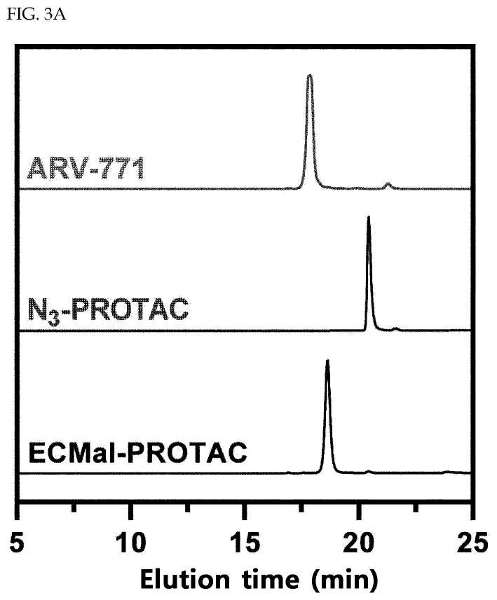

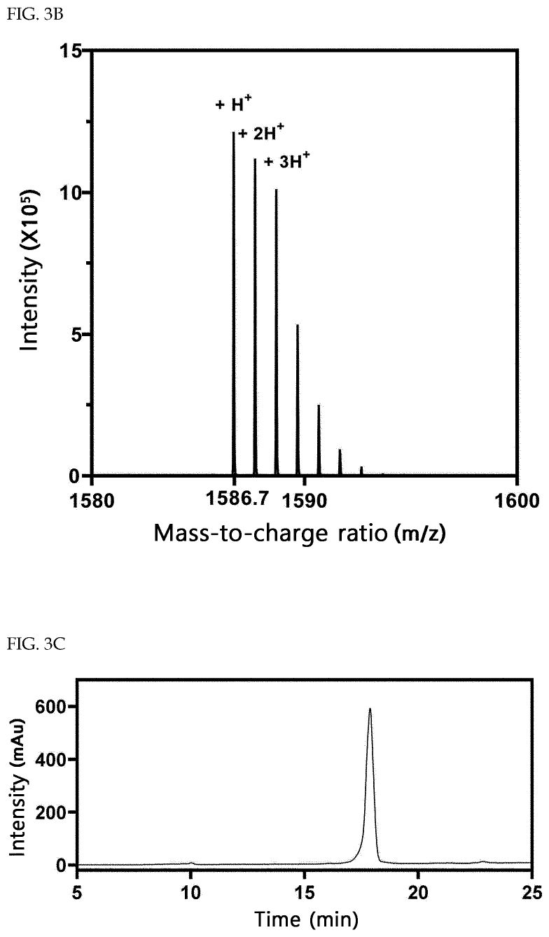

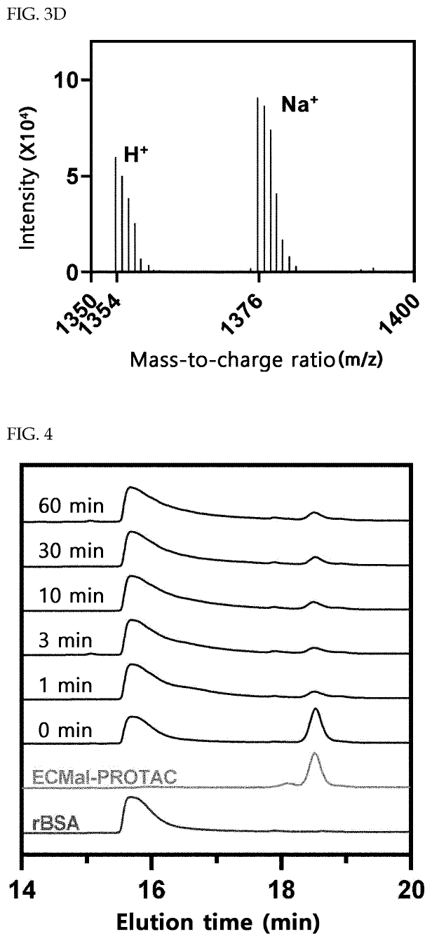

For the synthesis of ECMal-PROTAC, N₃-PROTAC (20.0 mg, 17.99 μmol) was placed in a 3 ml 1-neck round-bottom flask and dissolved in 0.5 ml of DMSO. BCN-PEG2-Mal (12.8 mg, 26.98 μmol) was added to the solution, and reacted at room temperature for 2 hours. The crude product was passed through the C18 preparative column of RP-HPLC. At this time, a gradient elution of ACN/H2O from 20/80 to 80/20 (v/v) was used, and the pure ECMal-PROTAC powder was lyophilized for separation. The products obtained at each step were confirmed through RP-HPLC and LC-MS analysis. The results of RP-HPLC and LC-MS analysis confirmed that the high-purity synthesis of ECMal-PROTAC was successful (N₃-PROTAC: yield 88.7%, purity 98.9%, ECMal-PROTAC: 62.9% yield and 98.2% purity) (FIG. 3A, 3B). Looking at FIG. 3A, each spectrum shows a complete shift of the corresponding peak for each reaction, indicating that the chemical conjugation was fully and successfully performed without significant side effects. Looking at FIG. 3B, the molecular weight measured through the LC-MS spectrum was found to be similar to the theoretical value (m/z=1586.7, [M]+1H⁺).

### Example 2. Synthesis of Mal-PROTAC

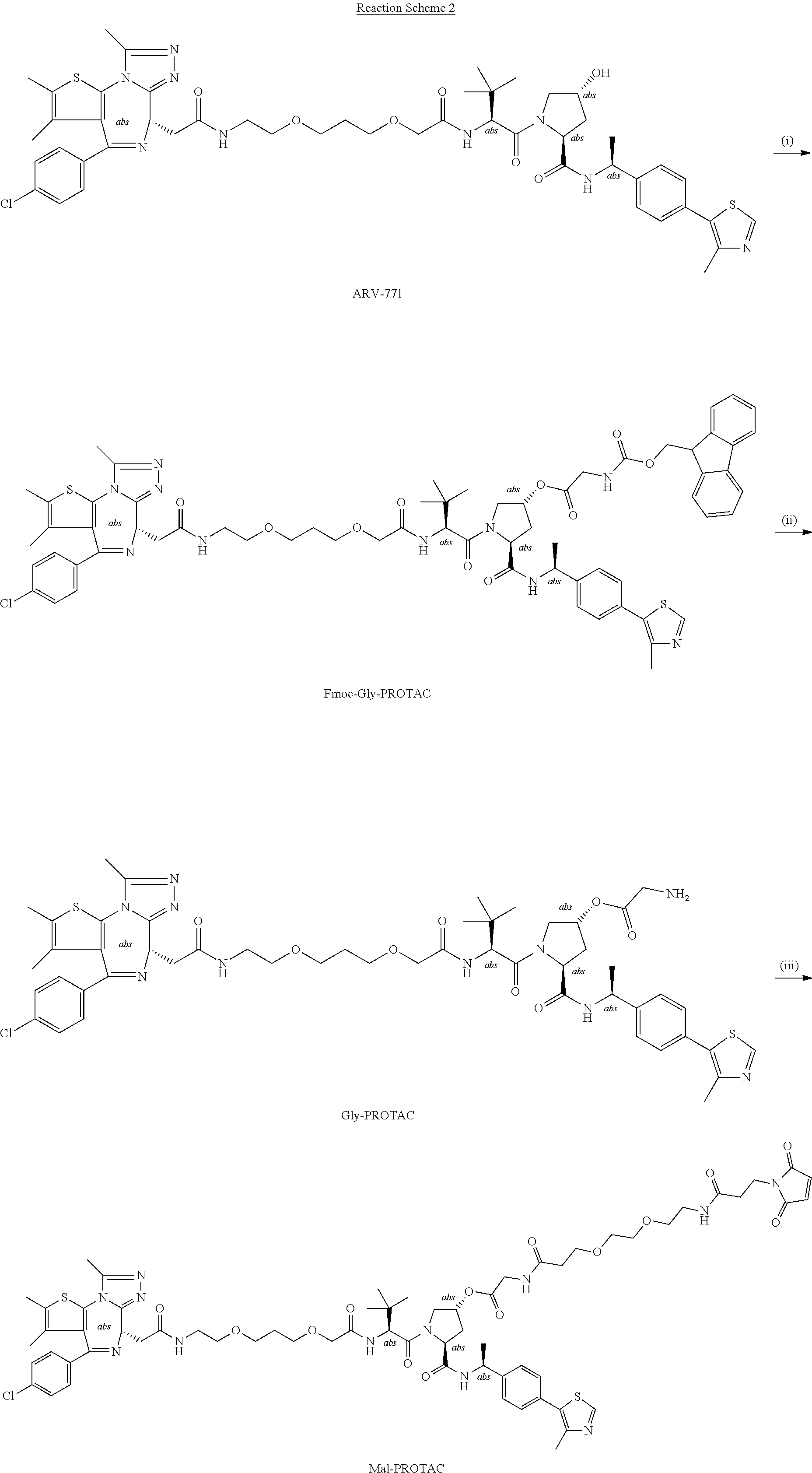

ARV-771 (20.0 mg, 20.27 μmol) was placed in a 3 ml 1-neck round-bottom flask equipped with a magnetic stirrer bar, and 0.5 ml of anhydrous DMF was added to dissolve it. After successfully adding Fmoc-Gly-OH (12.1 mg, 40.54 μmol), DMAP (7.43 mg, 60.81 μmol), and EDC-HCl (9.44 mg, 60.81 μmol) to the flask, the mixture was slowly mixed and reacted at room temperature for 12 hours. As a result, the crude product was concentrated under reduced pressure, and separated through C18 column chromatography (Sep-Pak C18 cartridges 5 g; Waters Corporation, Milford, MA, USA) using eluent of ACN/H2O (70/30, v/v) to obtain Fmoc-Gly-PROTAC. The recovered Fmoc-Gly-PROTAC was lyophilized to remove residual solvent.

To remove the Fmoc protecting group, Fmoc-Gly-PROTAC (20.0 mg, 15.80 μmol) was placed in a 3 ml 1-neck round-bottom flask equipped with a magnetic stirrer bar, and dissolved by adding 0.5 ml of a 20% (v/v) piperidine/DMF mixed solution. It was then reacted at room temperature for 15 minutes. As a result, the crude product was concentrated under reduced pressure, and separated through C18 column chromatograpy (Sep-Pak C18 cartridges 5 g; Waters Corporation, Milford, MA, USA) using eluent of ACN/H2O (50/50, v/v) to obtain Gly-PROTAC. The recovered Fmoc-Gly-PROTAC was lyophilized to remove residual solvent.

To synthesize Mal-PROTAC, Gly-PROTAC (15.0 mg, 14.37 μmol) was placed in a 3 ml 1-neck round-bottom flask equipped with a magnetic stirrer bar, and 0.5 ml of anhydrous DMF was added to dissolve it. After successfully adding Maleimide-PEG2-NHS Ester (12.23 mg, 28.74 μmol) and DIPEA (7.51 μl, 43.12 μmol) to the solution, it was reacted at room temperature for 12 hours. As a result, the crude product was concentrated under reduced pressure and passed through the C18 preparative column of RP-HPLC. At this time, a gradient elution of ACN/H2O from 20/80 to 80/20 (v/v) was used, and the pure Mal-PROTAC powder was lyophilized for separation.

### Experimental Example 1. In Vitro Binding Rate of ECMal-PROTAC Prepared from Example 1 and Albumin

The binding rate of ECMal-PROTAC from example 1 and albumin was confirmed through RP-HPLC. Specifically, a BSA solution (200 μM) diluted with PBS was co-incubated by mixing it with an ECMal-PROTAC aqueous solution (140 μM) of example 1 at a 1:1 volume ratio. The culture was collected at incubation times of 0 minutes, 5 minutes, 30 minutes, and 60 minutes. It was quenched with mercaptoethanol, and HPLC spectra for each sample were acquired through concentration gradient of the eluent (ACN/H2O, 20/80 to 80/20 (v/v), 30 minutes).

To verify the albumin binding ability of the complex for cancer targeting (ECMal-PROTAC) of example 1, the mixture of BSA and the complex for cancer targeting (ECMal-PROTAC) was analyzed by LC-MS spectrum, and compared with the LC-MS spectrum of free BSA.

BSA-bound ECMal-PROTAC (Alb-ECMal-PROTAC) is formed in the mixture of BSA and the complex for cancer targeting (ECMal-PROTAC) of example 1 through a thiol-maleimide reaction. Since almost all of the complex for cancer targeting binds to albumin to form Alb-ECMal-PROTAC, BSA and the complex for cancer targeting (ECMal-PROTAC) mixture and Alb-ECMal-PROTAC may be used interchangeably in some experimental examples.

FIG. 4 is the HPLC spectrum of a mixture of BSA and the complex for cancer targeting (ECMal-PROTAC). According to this, it can be seen that BSA and the complex for cancer targeting (ECMal-PROTAC) rapidly bind through a thiol-maleimide click chemical reaction. To confirm the albumin binding ability of the complex for cancer targeting (ECMal-PROTAC) of example 1, bovine serum albumin (BSA), which has a structure similar to human serum albumin (HSA), was used for analysis. As a result of mixing the complex for cancer targeting (ECMal-PROTAC) of example 1 with BSA and analyzing it over time by RP-HPLC, it was confirmed that the peak of the complex for cancer targeting (ECMal-PROTAC) rapidly decreased and the peak of BSA gradually broadened.

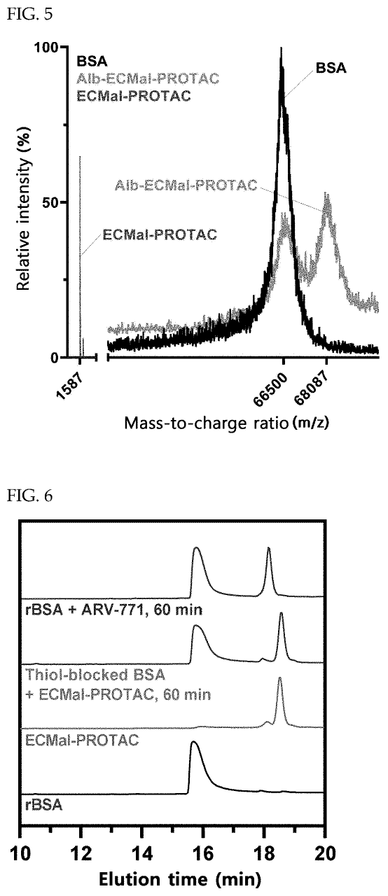

FIG. 5 is the LC-MS spectrum for a mixture of BSA and the complex for cancer targeting (ECMal-PROTAC). BSA alone and the complex for cancer targeting (ECMal-PROTAC) alone were also analyzed by LC-MS and shown in FIG. 5.

According to FIG. 5, the change in molecular weight of the complex for cancer targeting (ECMal-PROTAC) according to example 1 and Alb-ECMal-PROTAC was confirmed to be 1,587 Da. This was confirmed to be consistent with the molecular weight change of the complex for cancer targeting (ECMal-PROTAC) according to example 1. That is, it can be seen that the complex for cancer targeting (ECMal-PROTAC) according to example 1 binds safely to albumin without chemical or physical change.

### Experimental Example 2. In Vitro Selective Binding Characteristics of ECMal-PROTAC Prepared from Example 1 and Albumin

To confirm whether the binding of ECMal-PROTAC of example 1 and albumin is specific, thiol-blocked BSA was used. Specifically, thiol-blocked BSA was prepared by mixing 4-maleimidobutyric acid and BSA with a cyc34 blocking agent. The thiol-blocked BSA was diluted with PBS to prepare a solution (200 μM). This was co-incubated by mixing it with the ECMal-PROTAC aqueous solution (140 μM) of example 1 at a 1:1 volume ratio. The culture was collected at incubation times of 0 minutes, 5 minutes, 30 minutes, and 60 minutes. It was quenched with mercaptoethanol, and HPLC spectra for each sample were acquired through concentration gradient of the eluent (ACN/H2O, 20/80 to 80/20 (v/v), 30 minutes).

At this time, the HPLC spectra were also analyzed for a co-incubation of ARV-771 (140 μM) and BSA solution (200 μM) mixed at a 1:1 volume ratio as a comparative example, and for the ECMal-PROTAC solution and BSA solution alone without any treatment of example 1.

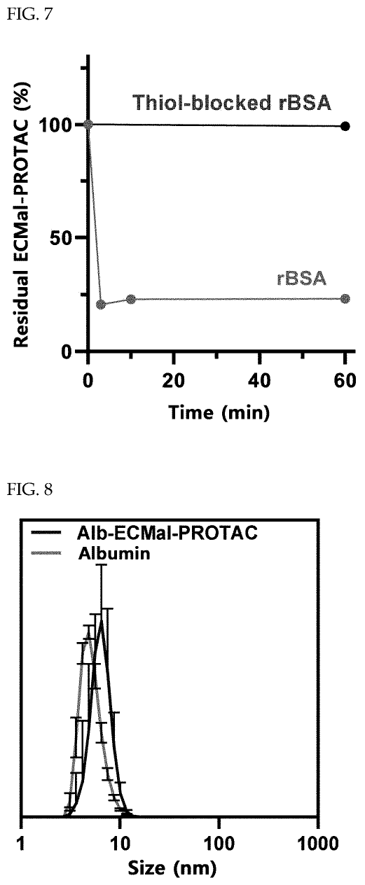

From the HPLC results for the BSA and complex for cancer targeting (ECMal-PROTAC) mixture in Experimental Example 1 and the thiol-blocked BSA and complex for cancer targeting (ECMal-PROTAC) mixture in Experimental Example 2, the reduction in the HPLC peak area associated with the complex for cancer targeting (ECMal-PROTAC) was quantified. This was calculated through changes in integrities and shown in FIG. 7.

FIG. 6 is the HPLC spectrum for a mixture of BSA and ARV-771, a mixture of thiol-blocked BSA and the complex for cancer targeting (ECMal-PROTAC), the complex for cancer targeting (ECMal-PROTAC), and BSA. FIG. 7 is a graph quantifying and showing the reduction in the HPLC peak area associated with the complex for cancer targeting (ECMal-PROTAC) from FIG. 4 and FIG. 6.

According to FIG. 6, it was confirmed that ARV-771 did not form an interaction even when mixed with BSA for more than 60 minutes. In addition, it was confirmed that the mixture of thiol-blocked BSA and the complex for cancer targeting (ECMal-PROTAC) also did not form an interaction. That is, it can be seen that the complex for cancer targeting (ECMal-PROTAC) of example 1 and BSA undergo a site-specific reaction.

According to FIG. 7, it was confirmed that the thiol-maleimide reaction of BSA and the complex for cancer targeting (ECMal-PROTAC) was completed by more than 75% within 3 minutes, and 100% completed within 10 minutes.

### Experimental Example 3. Particle Size Analysis of Alb-ECMal-PROTAC

A BSA solution (200 μM) diluted with PBS was co-incubated by mixing it with an ECMal-PROTAC aqueous solution (140 μM) of example 1 at a 1:1 volume ratio. The culture was collected after 30 minutes to obtain Alb-ECMal-PROTAC. The particle size of Alb-ECMal-PROTAC and BSA was measured through DLS.

FIG. 8 is the result of DLS analysis for the complex for cancer targeting (ECMal-PROTAC) according to example 1 before and after binding to albumin (BSA). According to this, no significant change in particle size was observed when ECMal-PROTAC bound to albumin. The average particle size of native BSA and Alb-ECMal-PROTAC was 5.43±1.42 and 6.99±1.38 nm, respectively. This shows that the binding of albumin and ECMal-PROTAC does not induce degradation or aggregation.

### Experimental Example 4. Confirmation of Whether the Complex for Cancer Targeting (ECMal-PROTAC) of Example 1 Degrades Under Esterase Conditions after Binding to Albumin

A BSA solution (200 μM) diluted with PBS was co-incubated by mixing it with an ECMal-PROTAC aqueous solution (140 μM) of example 1 at a 1:1 volume ratio. The culture was reacted at pH 8.0 condition for 30 minutes, and then collected to obtain Alb-ECMal-PROTAC. 15 units of esterase was added to 0.2 ml of the Alb-ECMal-PROTAC aqueous solution. The mixture was collected at reaction times of 0 hours, 3 hours, 6 hours, and 9 hours, and analyzed by RP-HPLC. Free ARV-771 was used as a control.

Since the complex for cancer targeting (ECMal-PROTAC) of example 1 is internalized into cancer cells in an albumin-bound form under physiological conditions, Alb-ECMal-PROTAC was used instead of the complex for cancer targeting (ECMal-PROTAC) of example 1.

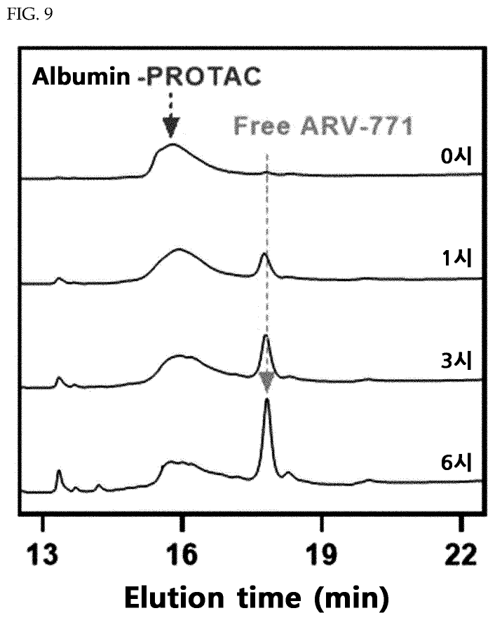

FIG. 9 is the result of HPLC analysis over time after treating Alb-ECMal-PROTAC with esterase (15 units). According to FIG. 9, it was confirmed that Alb-ECMal-PROTAC was cleaved at the ester bond by esterase, resulting in the gradual release of ARV-771. It was confirmed that Alb-ECMal-PROTAC released 63.2% ARV-771 within 48 hours. This example was performed in vitro. It is believed that the degradation rate will be accelerated by acidic pH in vivo. Furthermore, since BSA and HSA derived from mammalian plasma are structurally very similar, it is believed that they will have excellent binding and degradation effects in HSA as well.

### Experimental Example 5. In Vitro Analysis of Cellular Absorption Rate of ECMal-PROTAC of Example 1

The complex for cancer targeting (ECMal-PROTAC) of example 1 showed significantly enhanced cellular absorption rate through enhanced endocytosis. To confirm this, a Cy5.5-BSA solution (Cy5.5-Alb, Preparation Example 1, 200 μM) diluted with PBS was co-incubated by mixing it with an ECMal-PROTAC aqueous solution (140 μM) of example 1 at a 1:1 volume ratio. The culture was reacted at pH 8.0 condition for 30 minutes, and then collected to obtain Cy5.5-Alb-ECMal-PROTAC. Cy5.5-ARV-771 of Preparation Example 1 was used as a control group. A 4T1 mouse breast cancer cell line was prepared. 1×105 4T1 cells were dispensed into a glass-bottom confocal dish (35 mm diameter) and cultured for 24 hours in RPMI medium. After stabilization, the cells were treated with Cy5.5-ARV-771 (5 μM) or Cy5.5-Alb-ECMal-PROTAC (5 μM), and cultured at 37° C. for 1 hour, 3 hours, 6 hours, and 12 hours. The cells were washed 3 times with PBS, treated with a 4% formaldehyde aqueous solution for 15 minutes, and then the cell nucleus was stained with DAPI (4,6-diamidino-2-phenylindole) for 10 minutes. The DAPI and Cy5.5 fluorescence signals in the 4T1 cells were measured and analyzed by CLSM.

The cellular uptake of ARV-771 and Alb-ECMal-PROTAC over time was evaluated through the analysis results. The relative intracellular fluorescence intensity of FITC was calculated using Image-Pro software (Media Cybernetics, Rockville, MD, USA), and the change was expressed as a function of time.

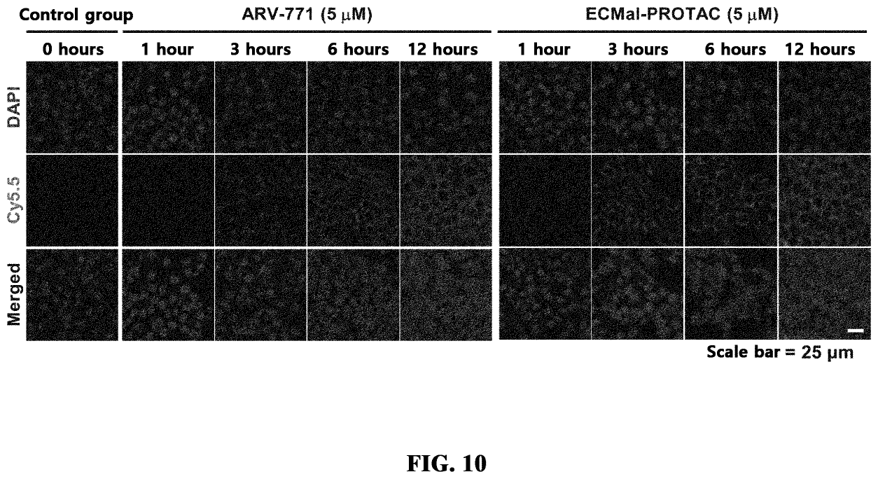

FIG. 10 is a confocal microscope image of 4T1 cancer cells treated with Cy5.5-ARV-771 or Cy5.5-Alb-ECMal-PROTAC. FIG. 11 is a graph quantifying and showing the intracellular fluorescence intensity in 4T1 cancer cells treated with Cy5.5-ARV-771 or Cy5.5-Alb-ECMal-PROTAC.

According to FIG. 10, it can be seen that both Cy5.5-ARV-771 and Cy5.5-Alb-ECMal-PROTAC were gradually absorbed into 4T1 cells within 12 hours. The intracellular absorbed concentrations of Cy5.5-ARV-771 and Cy5.5-Alb-ECMal-PROTAC were also confirmed to be similar (relative fluorescence intensities of 0.61±0.11 and 0.61±0.06, respectively). However, the cellular absorption rate of Cy5.5-Alb-ECMal-PROTAC was confirmed to be slightly faster than that of Cy5.5-ARV-711. Through this, it can be seen that the complex for cancer targeting (ECMal-PROTAC) of example 1 binds to albumin in vivo, and this structural feature leads to an inclination for active absorption into cancer cells.

### Experimental Example 6. In Vitro Therapeutic Effect of ECMal-PROTAC of Example 1

BRD4 is a protein that regulates gene transcription, and is known to be largely involved in the transcription of cancer-related genes. Therefore, if silencing of BRD4 occurs in cancer cells, cell apoptosis is induced by suppressing cancer gene transcription.

Accordingly, Western blot analysis was performed to confirm the anticancer ability by evaluating the presence or absence of degradation of BRD4 and other related proteins (e.g., c-Myc and Bcl-2) when cancer cells were treated with the complex for cancer targeting (ECMal-PROTAC) of example 1. The complex for cancer targeting (ECMal-PROTAC) of example 1 and ARV-771 were prepared as samples.

First, 1×105 4T1 cells were dispensed into each well of a 6-well culture plate and cultured for 24 hours at 37° C. and 5% carbon dioxide atmosphere. The cell culture medium was supplemented with various concentrations of ARV-771 (0 to 10 μM) or the complex for cancer targeting (ECMal-PROTAC) of example 1 (0 to 10 μM), and cultured at 37° C. for 24 hours. After the culture was completed, the cells were washed 3 times with PBS, and lysed with a lysis buffer containing 1% protease. Cell debris from the recovered lysate was removed by centrifugation at 12,000 rpm for 25 minutes. After recovering the supernatant, the protein amount was measured using a BCA kit. The supernatant was loaded onto a 10% sodium dodecyl sulfate-polyacrylamide gel. Electrophoresis (SDS-PAGE) was performed, followed by blotting onto a polyvinylidene difluoride (PVDF) membrane. After blotting, the PVDF membrane was treated with 5% BSA-containing tris buffered saline containing 0.1% Tween 20 (TBS-T) for 2 hours to suppress the non-specific binding of IgG (immunoglobulin G). It was immediately exposed to a rabbit anti-mouse primary antibody (1,000:1). After treating at 4° C. for 24 hours, the PVDF membrane was washed 3 times with TBS-T and further incubated with an HRP-conjugated mouse anti-rabbit IgG antibody at room temperature for 1 hour. The immunoreactive bands on the PVDF membrane were analyzed through an ECL (enhanced chemiluminescence) system.

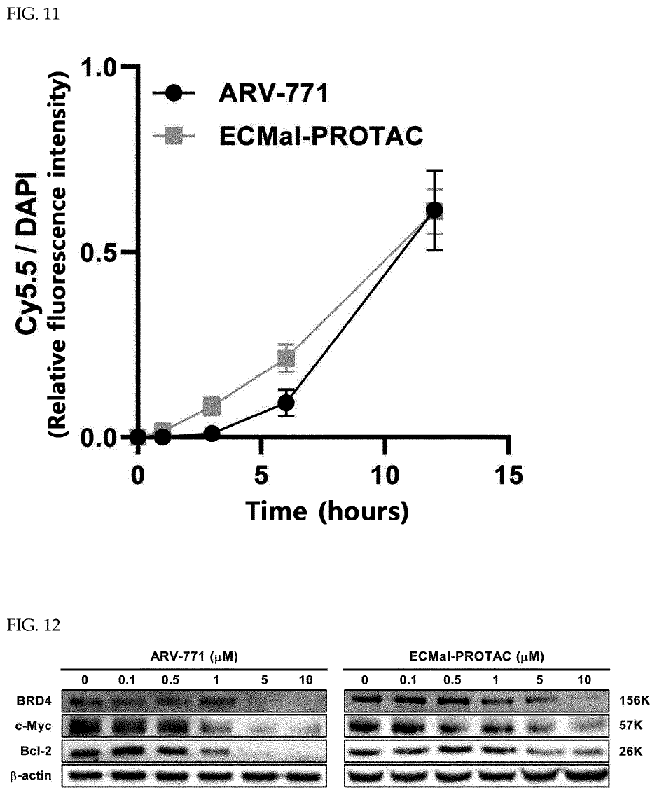

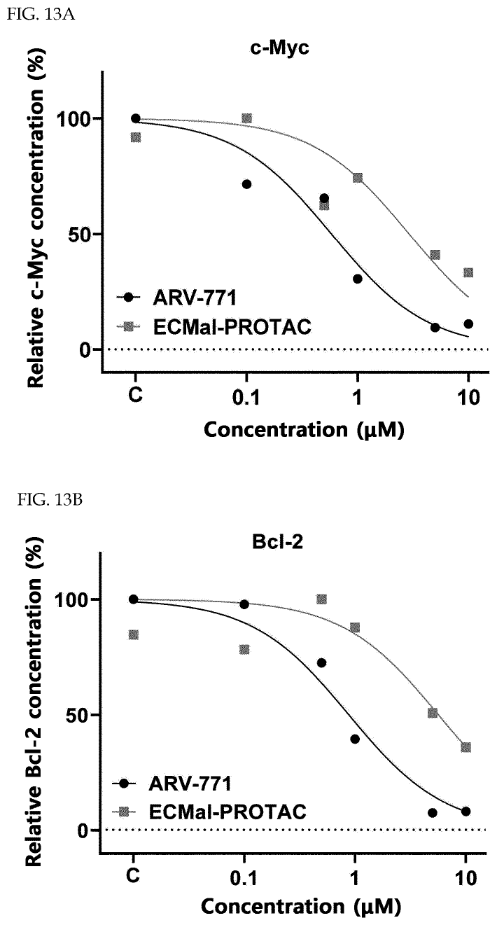

FIG. 12 is the result of Western blot analysis after treating 4T1 cancer cells with ARV-771 or the complex for cancer targeting (ECMal-PROTAC) of example 1. FIG. 13 is a graph quantifying the relative expression levels of c-Myc (a) and Bcl-2 (b) in 4T1 cancer cells treated with ARV-771 or the complex for cancer targeting (ECMal-PROTAC) of example 1. FIG. 14 is a graph quantifying the BRD4 expression level according to the concentration of ARV-771 or the complex for cancer targeting (ECMal-PROTAC) of example 1.

According to FIG. 12 to 14, it was confirmed that the complex for cancer targeting (ECMal-PROTAC) of example 1 binds to MSA (mouse serum albumin) in the cell culture medium, is efficiently internalized into 4T1 cells, and degrades the overexpressed BRD4 protein in a concentration-dependent manner. Therefore, it can be seen that the complex for cancer targeting (ECMal-PROTAC) of example 1 is successfully degraded by esterase present inside the cancer cells, releasing ARV-771.

According to FIGS. 13A to 13B, it was confirmed that the complex for cancer targeting (ECMal-PROTAC) of example 1 also reduces the levels of other proteins such as c-Myc and Bcl-2. C-Myc and Bcl-2 play an important role in the mRNA transcription of BRD4, indicating that they are effectively involved in BRD4 degradation.

According to FIG. 14, quantification of the BRD4 band confirmed that the DC50 (half-maximal degradation concentration) of the complex for cancer targeting (ECMal-PROTAC) of example 1 was 1.81 μM. This level is similar to ARV-771 (DC50=3.38 μM).

Summarizing the above results, it was confirmed that the complex for cancer targeting (ECMal-PROTAC) of example 1 is successfully absorbed into cancer cells through endocytosis. The ECMal linker is efficiently cleaved by esterase present inside the cancer cells, releasing PROTAC. This leads to the induction of apoptosis in cancer cells.

### Experimental Example 7. In Vitro Therapeutic Effect of the Complex for Cancer Targeting (ECMal-PROTAC) of Example 1 on Cancer Cells

The toxicity of ARV-771 and ECMal-PROTAC on cancer cells was analyzed by the CCK-8 (cell counting kit-8) assay. 4T1 cells were dispensed into a 96-well culture plate at 3×103 cells per well. They were cultured for 24 hours at 37° C. and 5% carbon dioxide atmosphere. The cells were treated with different concentrations of ARV-771 (0.01 to 100 μM), the complex for cancer targeting (ECMal-PROTAC) of example 1 (0.01 to 100 μM), and the complex for cancer targeting (Mal-PROTAC) of example 2 (0.01 to 100 μM) separately, and cultured for 48 hours. After exposing each well to 10% CCK-8-containing culture media for 30 minutes, the viability of 4T1 cells treated with ARV-771 or 4T1 cells treated with ECMal-PROTAC or Mal-PROTAC was analyzed with a microplate reader. The change in cell viability according to the concentration of ARV-771 or ECMal-PROTAC or Mal-PROTAC was calculated using GraphPad Prism 8 software (GraphPad Software, San Diego, CA, USA) by fitting the IC50 (haf-maximal inhibitory concentration) value.

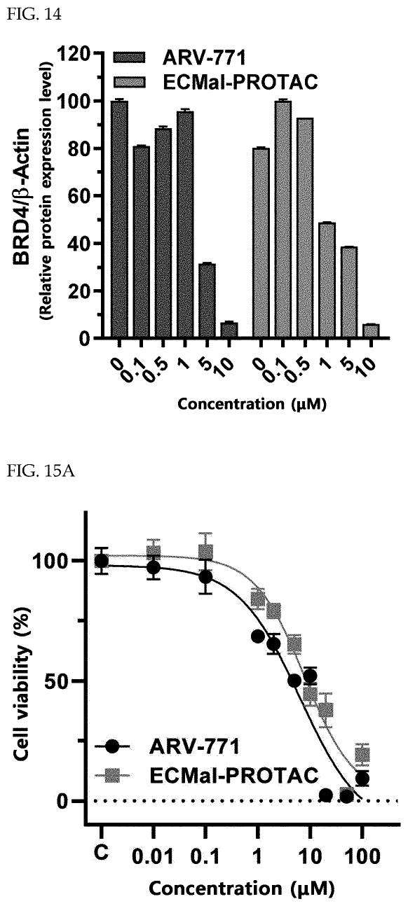

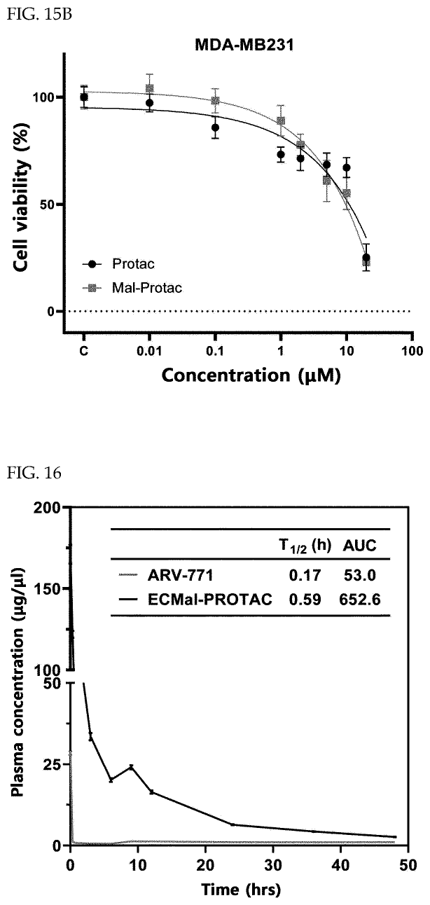

FIGS. 15A and 15B are graphs analyzing the survival rate after treating 4T1 cancer cells or MDA-MB231 with the complex for cancer targeting (ECMal-PROTAC) of example 1 (FIG. 15A) and the complex for cancer targeting (Mal-PROTAC) of example 2 (FIG. 15B).

According to FIGS. 15A to 15B, the complex for cancer targeting (ECMal-PROTAC) of example 1 was confirmed to have an IC50 of 7.780 μM against cancer cells. That is, the complex for cancer targeting according to the present invention has remarkable anti-proliferative properties against cancer cells rich in BRD4.

When the complex for cancer targeting (ECMal-PROTAC) of example 1 and the complex for cancer targeting (Mal-PROTAC) of example 2 are internalized into cells, they induce BRD4 deficiency and reduced expression of c-Myc and Bcl-2, which are downstream effectors of BRD4. Through this, they promote apoptosis of cancer cells. The complex for cancer targeting (ECMal-PROTAC, Mal-PROTAC) of example 1 and example 2 exists in vivo in an inactivated state by binding to albumin in vivo. It is believed that this causes a slight increase in IC50 compared to native ARV-771 (IC50=6.375 μM). However, it is still a significant effect for anticancer treatment. Therefore, it can be seen that the complex for cancer targeting (ECMal-PROTAC, Mal-PROTAC) according to the present invention promotes BRD4 degradation and successfully recruits the VHL E3 pathway to induce excellent cancer cell apoptosis, and thus can be easily utilized as an anticancer agent.

### Experimental Example 8. In Vivo Analysis of Biodistribution and Cancer Targeting Potential of the Complex for Cancer Targeting (ECMal-PROTAC) of Example 1

The in vivo pharmacokinetic properties and biodistribution of ARV-771 and the complex for cancer targeting (ECMal-PROTAC) of example 1 were investigated.

### Cancer Animal Model

5-week-old female Balb/c and Balb/c nu/nu mice were purchased from NaraBio, Inc (Seoul, Republic of Korea). The mice underwent a 2-week acclimatization period before the example. The mice were raised under free-feeding conditions in a breeding room maintained at a temperature of 22±2° C. and humidity of 40-60% during the example. The light and dark cycle was regulated at 12-hour intervals. All examples were conducted in compliance with the relevant laws and institutional guidelines of the KIST Institutional Animal Care and Use Committee (IACUC; Approval No.: 2021-143).

Cancer animal models were prepared by inoculating 1×106 4T1 cells into the left thigh of the mice, and raising them until the tumor volume reached 150-200 mm3.

### Preparation of Samples

Samples were prepared as follows to analyze the in vivo therapeutic effect of the complex for cancer targeting (ECMal-PROTAC) of example 1. A Cy5.5-BSA solution (Cy5.5-Alb, Preparation Example 1, 200 μM) diluted with PBS was co-incubated by mixing it with an ECMal-PROTAC aqueous solution (140 μM) of example 1 at a 1:1 volume ratio. The culture was reacted at pH 8.0 condition for 30 minutes, and then collected to obtain Cy5.5-Alb-ECMal-PROTAC. Cy5.5-ARV-771 of Preparation Example 1 was used as a control group.

### Administration of Samples

For the preparation of the control group and the example group, mice were randomly divided into groups of 3.

The comparison group used Cy5.5-ARV-771 (3 mg/kg, equiv. to ARV-771) as the sample. The example group used Cy5.5-Alb-ECMal-PROTAC (3 mg/kg, equiv. to ARV-771) as the sample. The control group used saline (3 mg/kg) as the sample. The Cy5.5-ARV-771 and Cy5.5-Alb-ECMal-PROTAC were administered through the tail vein.

### Analysis

To investigate the change in blood circulation level over time from each group, whole blood was collected at specific time points (0 to 96 hours). The collected blood samples were analyzed for fluorescence intensity using the IVIS Lumina Series III system. The data was expressed as a function of time with GraphPad Prism 8 software to calculate the blood half-life and AUC (the area under the curve).

Furthermore, to analyze the degree of tumor accumulation, whole-body fluorescence images of the control group and the example group were taken using the IVIS Lumina Series III system at 0, 1, 3, 6, 12, and 24 hours after sample administration.

The control group and the example group were euthanized 24 hours after administration, and major organs (heart, liver, lung, spleen, and kidney) and cancer tissue were excised. The biodistribution and histological analysis of the excised tissues were performed as follows.

To analyze the sample distribution in the excised cancer tissue, the excised major organs and cancer tissue were imaged with the IVIS Lumina Series III system (PerkinElmer, Waltham, MA, USA) and analyzed with Living Image software (PerkinElmer).

For histological analysis of the excised cancer tissue, the separated cancer tissue was immersed in 4% formaldehyde solution, fixed at 4° C., and then sliced into sections of 1-2 mm3 in size. Each section was shaken and incubated with TBST containing 3% BSA for 3 hours. FITC-conjugated anti-CD31 primary antibody (Biolegend, USA) was added and stained by incubation overnight at 4° C. After washing 3 times with TBST, it was placed on a glass cover slide in DAPI mounting medium, and imaged with a confocal laser scanning microscope (CLSM; Leica TCS SP8, Leica Microsystems GmbH, Wetzlar, Germany). The imaged images were quantified using a microplate reader (VERSAmax™, Molecular Devices Corp., Sunnyvale, CA, USA).

### Result Analysis

FIG. 16 is a pharmacokinetic profile analyzing the plasma concentration over time for the comparison group (Cy5.5-ARV-771 intravenous injection) and the example group (Cy5.5-Alb-ECMal-PROTAC intravenous injection). According to this, it was confirmed that the blood half-life of the complex for cancer targeting (ECMal-PROTAC) of example 1 was significantly increased when formed as Cy5.5-Alb-ECMal-PROTAC through binding to albumin. Specifically, as a result of observing the fluorescence intensity of Cy5.5-ARV-771 and Cy5.5-Alb-ECMal-PROTAC from the plasma of the comparison group and the example group, Cy5.5-Alb-ECMal-PROTAC remained in the plasma until 12 hours, and Cy5.5-ARV-771 was completely removed after 1 hour. That is, the half-life of Cy5.5-Alb-ECMal-PROTAC was 0.59 hours, which was confirmed to be 3.5 times greater than Cy5.5-ARV-771. Furthermore, the AUC of Cy5.5-Alb-ECMal-PROTAC was confirmed to be 12 times greater than Cy5.5-ARV-771. Therefore, the plasma retention time of the complex for cancer targeting (ECMal-PROTAC) according to the present invention is confirmed to be significantly longer than conventional anticancer agents. The complex for cancer targeting (ECMal-PROTAC) according to the present invention improved the problems (hydrophobicity and low water solubility) that the PROTAC molecule had through in vivo albumin binding.

In the graphs including FIG. 16, ‘hr’ or ‘hrs’ refers to hours, which is a unit of period of time. Therefore, 1 hr or 1 hour primarily means ‘60 minutes’. However, ‘hr(s)’ and ‘hour(s)’ may be used interchangeably in some specifications, and in this case, the meaning can be inferred from the context.

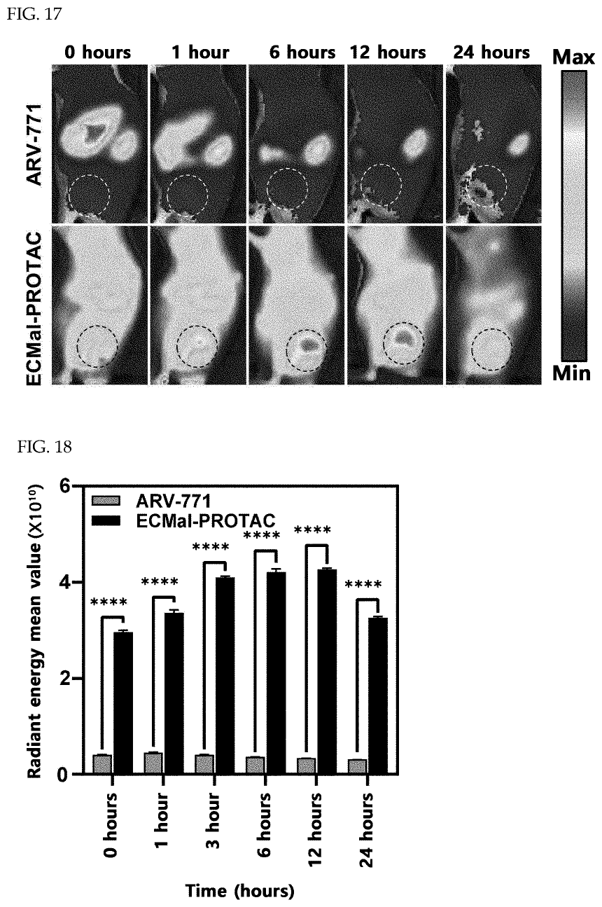

FIG. 17 is an NIRF (non-invasive near-infrared fluorescence) image over time for the comparison group (Cy5.5-ARV-771 intravenous injection) and the example group (Cy5.5-Alb-ECMal-PROTAC intravenous injection).

According to FIG. 17, the example group (Cy5.5-Alb-ECMal-PROTAC intravenous injection) was specifically accumulated in the cancer tissue, and this concentration was confirmed to be significantly and remarkably higher than the comparison group (Cy5.5-ARV-771 intravenous injection).

FIG. 18 is a graph quantifying only the fluorescence signal in the cancer tissue from the results of the comparison group (Cy5.5-ARV-771 intravenous injection) and the example group (Cy5.5-Alb-ECMal-PROTAC intravenous injection) in FIG. 17 (****p<0.0001). According to this, Cy5.5-Alb-ECMal-PROTAC was confirmed to reach the highest level 12 hours after administration. The fluorescence intensity was 12.6 times higher for Cy5.5-Alb-ECMal-PROTAC than for Cy5.5-ARV-771.

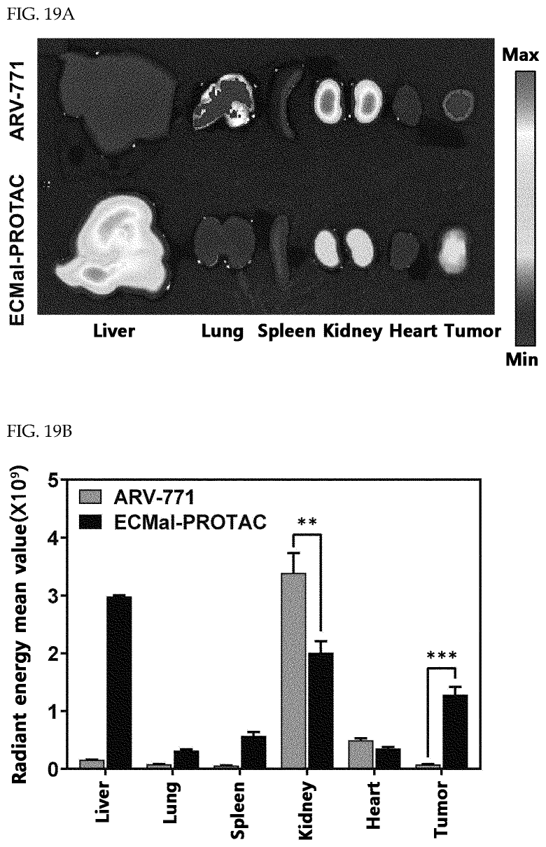

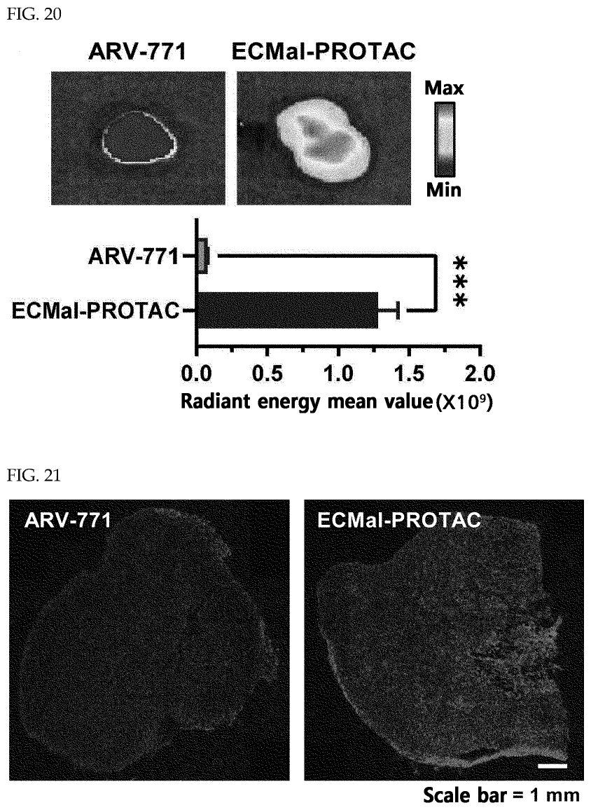

FIGS. 19A and 19B are the ex vivo fluorescence images (FIG. 19A) and quantified fluorescence intensity graph (FIG. 19B) of major organs (liver, lung, spleen, kidney, heart) and cancer tissue harvested from the comparison group (Cy5.5-ARV-771 intravenous injection) and the example group (Cy5.5-Alb-ECMal-PROTAC intravenous injection). FIG. 20 is the fluorescence image (upper) and quantified fluorescence intensity graph (lower) of cancer tissue harvested from the comparison group (Cy5.5-ARV-771 intravenous injection) and the example group (Cy5.5-Alb-ECMal-PROTAC intravenous injection) (***p<0.001).

According to FIG. 19 and FIG. 20, the comparison group administered with Cy5.5-ARV-771 had a very strong fluorescence signal in the kidney tissue. It had a very weak fluorescence signal in the cancer tissue and other organ tissues. This shows that a significant portion of Cy5.5-ARV-771 is immediately eliminated through the kidneys.

In contrast, the example group administered with Cy5.5-Alb-ECMal-PROTAC showed a strong fluorescence signal in the liver tissue and cancer tissue. In particular, the fluorescence intensity in the cancer tissue was confirmed to be 16.3 times higher compared to Cy5.5-ARV-771. That is, the complex for cancer targeting (ECMal-PROTAC) according to the present invention not only has a longer in vivo retention period than conventional PROTACs but also has high stability, very low probability of kidney excretion, and excellent cancer tissue targeting ability.

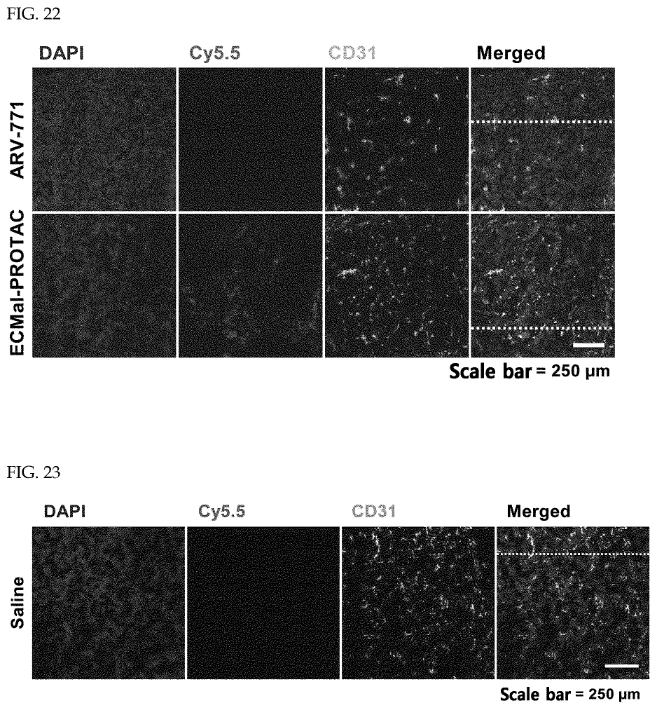

FIG. 21 is an enlarged fluorescence image of cancer tissue excised from the comparison group (Cy5.5-ARV-771 intravenous injection) and the example group (Cy5.5-Alb-ECMal-PROTAC intravenous injection).

According to FIG. 21, the cancer tissue of the example group was confirmed to have a uniform and abundant distribution of Cy5.5-Alb-ECMal-PROTAC throughout the tissue. In contrast, Cy5.5-ARV-771 was not evenly distributed throughout the cancer tissue in the comparison group. In particular, fluorescence was hardly observed inside the cancer tissue. That is, the complex for cancer targeting (ECMal-PROTAC) according to the present invention penetrates sufficiently in large quantities even into the central tissue of the cancer, thereby inducing an anticancer effect throughout the entire cancer tissue. Conventional anticancer agents required excessive doses or excessive treatment periods for complete cancer apoptosis and treatment because the drug efficacy could not be delivered to the dense interior of the cancer tissue. However, the complex for cancer targeting (ECMal-PROTAC) according to the present invention can achieve a significantly unpredictable pharmacological effect compared to conventional anticancer agents because it is easily delivered even to the center of the cancer tissue, even when the same dose is used.

FIG. 22 is an image taken with a confocal laser scanning microscope after staining cancer tissue excised from the comparison group (Cy5.5-ARV-771 intravenous injection) and the example group (Cy5.5-Alb-ECMal-PROTAC intravenous injection) with DAPI and the CD31 marker. FIG. 23 is an image taken with a confocal laser scanning microscope after staining cancer tissue of the control group (Saline, control) administered with saline instead of the sample, with DAPI and the CD31 marker.

According to FIG. 22 and FIG. 23, the cancer tissue of the example group administered with Cy5.5-Alb-ECMal-PROTAC delivered a larger amount of Cy5.5-Alb-ECMal-PROTAC to the cancer. It was confirmed that the delivered drugs completely penetrated not only the cancer near the blood vessels but also the tissue inside. Furthermore, a large amount of Cy5.5-Alb-ECMal-PROTAC was confirmed to be absorbed and accumulated even inside the cells.

That is, it can be seen that the complex for cancer targeting (ECMal-PROTAC) according to the present invention is delivered deeply into not only the cancer near the blood vessels but also the cells inside the cancer tissue. Therefore, it will exhibit a significantly unpredictable anticancer effect that could not be obtained with conventional anticancer agents.

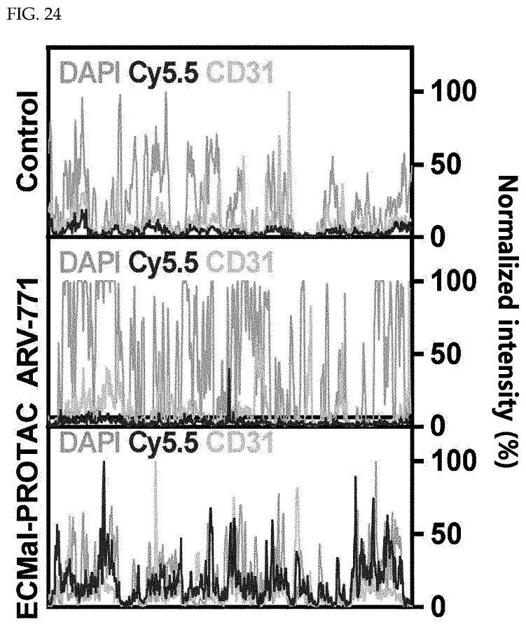

FIG. 24 is a graph diagramming the fluorescence signal on the cross-section of cancer tissue excised from the control group (control, Saline), the comparison group (Cy5.5-ARV-771 intravenous injection), and the example group (Cy5.5-Alb-ECMal-PROTAC intravenous injection).

According to FIG. 24, a significant difference appeared between the Cy5.5 and CD31 signals in the cancer tissue of the example group. No significant difference was confirmed between the Cy5.5 and CD31 signals in the cancer tissue of the control group and the comparison group. That is, the complex for cancer targeting (ECMal-PROTAC) according to the present invention has a long blood retention time, specifically accumulates in cancer tissue, and the drug penetrates broadly and uniformly into the cancer tissue in addition to the blood vessel tissue. This is a significant and remarkable difference compared to conventional anticancer agents.

### Experimental Example 9. Analysis of Anticancer Efficacy of the Complex for Cancer Targeting (ECMal-PROTAC) on Cancer Animal Model

The in vivo cancer treatment efficacy of ARV-771 and the complex for cancer targeting (ECMal-PROTAC) of example 1 was investigated.

### Cancer Animal Model

5-week-old female Balb/c and Balb/c nu/nu mice were purchased from NaraBio, Inc (Seoul, Republic of Korea). The mice underwent a 2-week acclimatization period before the example. The mice were raised under free-feeding conditions in a breeding room maintained at a temperature of 22±2° C. and humidity of 40-60% during the example. The light and dark cycle was regulated at 12-hour intervals. All examples were conducted in compliance with the relevant laws and institutional guidelines of the KIST Institutional Animal Care and Use Committee (IACUC; Approval No.: 2021-143).

Cancer animal models were prepared by inoculating 1×10⁶ 4T1 cells into the left thigh of the mice, and raising them until the tumor volume reached 50-80 mm³.

### Administration of Samples

For the preparation of the control group, the comparison group, and the example group, mice were randomly divided into groups of 5.

The control group was administered saline (10 mg/kg) once every 3 days for 15 days through the tail vein (total 4 administrations). The comparison group was administered ARV-771 (10 mg/kg) once every 3 days for 15 days through the tail vein (total 4 administrations). The example group was administered ECMal-PROTAC 10 mg/kg (wquiv. to ARV-771) of example 1 once every 3 days for 15 days through the tail vein (total 4 administrations).

### Analysis

Tumor size and body weight were measured every two days for the control group, the comparison group, and the example group.

The control group, the comparison group, and the example group were euthanized after 15 days, and major organs (heart, liver, lung, spleen, kidney) and cancer tissue were excised.

To confirm whether the complex for cancer targeting (ECMal-PROTAC) of example 1 exhibits cancer-specific BRD4 degradation activity in vivo, cancer tissue was excised from the control group, the comparison group, and the example group, and BRD4-related gene expression was analyzed by Western blot from this tissue.

Furthermore, to analyze cancer cell apoptosis, TUNEL (terminal deoxynucleotidyl transferase dUTP nick end labeling) analysis was performed on cancer tissue excised from the control group, the comparison group, and the example group. Next, histological analysis was performed by H&E (hematoxylin & eosin) staining of the major organ tissues and cancer tissue of each excised group.

### Analysis Results

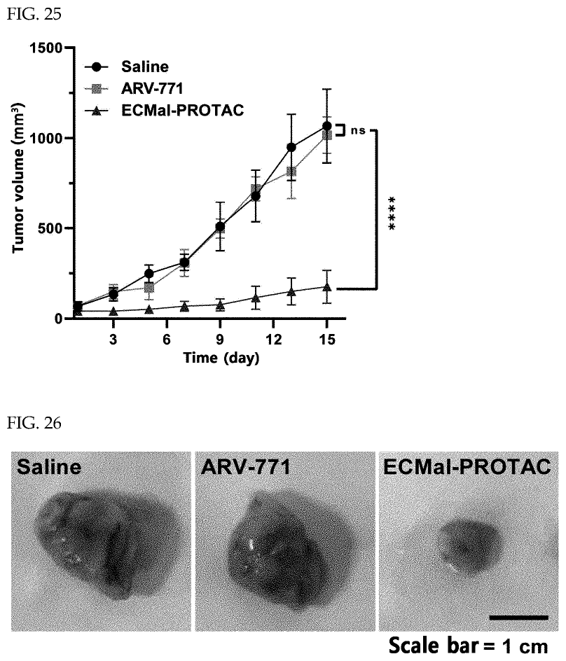

FIG. 25 is a graph measuring and showing the cancer size measured from the control group, the comparison group, and the example group over time (ns is indicated if no significant difference was shown. ****p<0.0001).

According to FIG. 25, the cancer size of the example group administered with the complex for cancer targeting (ECMal-PROTAC) of example 1 was confirmed to decrease significantly. The cancer size of the control group and the comparison group increased by 15.8 times and 14.8 times, respectively, from Day 0. The two groups were confirmed to show no significant difference. In contrast, the increase in cancer size was significantly reduced in the example group administered with the complex for cancer targeting (ECMal-PROTAC) of example 1. The tumor volume in the example group administered with the complex for cancer targeting (ECMal-PROTAC) of example 1 was confirmed to be 176.13±81.62 mm3 after 15 days of administration. This was confirmed to be 6.1 times and 5.3 times smaller than the control group and the comparison group, respectively.

ARV-771 and ECMal-PROTAC showed similar levels of toxicity to cancer cells in the in vitro example. However, in vivo, ECMal-PROTAC was confirmed to have an anti-tumor efficacy 5 to 6 times higher than ARV-771.

FIG. 26 is an optical image of cancer tissue excised from the control group, the comparison group, and the example group. FIG. 27 is a confocal laser scanning image taken after TUNEL staining of cancer tissue excised from the control group, the comparison group, and the example group.

According to FIG. 26 and FIG. 27, the example group administered with the complex for cancer targeting (ECMal-PROTAC) of example 1 was confirmed to have a much smaller tumor size than the other groups. Furthermore, the example group administered with the complex for cancer targeting (ECMal-PROTAC) of example 1 confirmed that cancer cell apoptosis was induced throughout the entire cancer tissue.

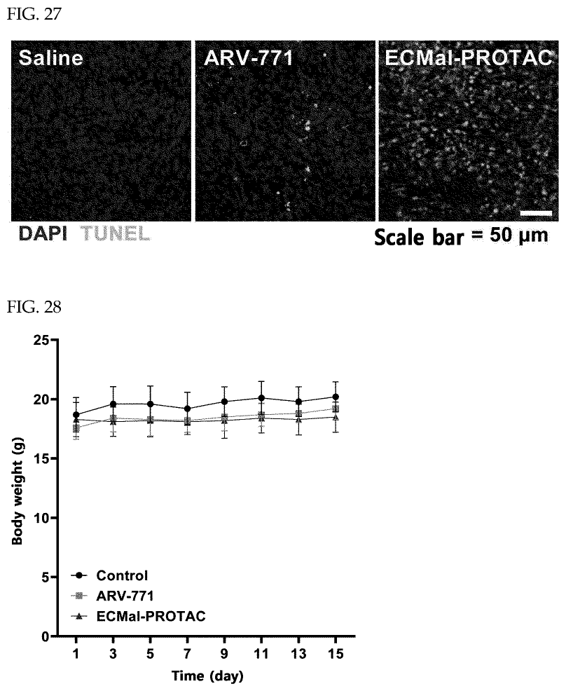

FIG. 28 is a graph analyzing and showing the weight change of the control group, the comparison group, and the example group. According to this, both the example group and the comparison group administered with the complex for cancer targeting (ECMal-PROTAC) of example 1 or ARV-771 did not show serious systemic side effects, and no significant weight loss was observed. That is, it can be seen that the complex for cancer targeting (ECMal-PROTAC) of example 1 and ARV-771 are safe substances that do not cause side effects in vivo.

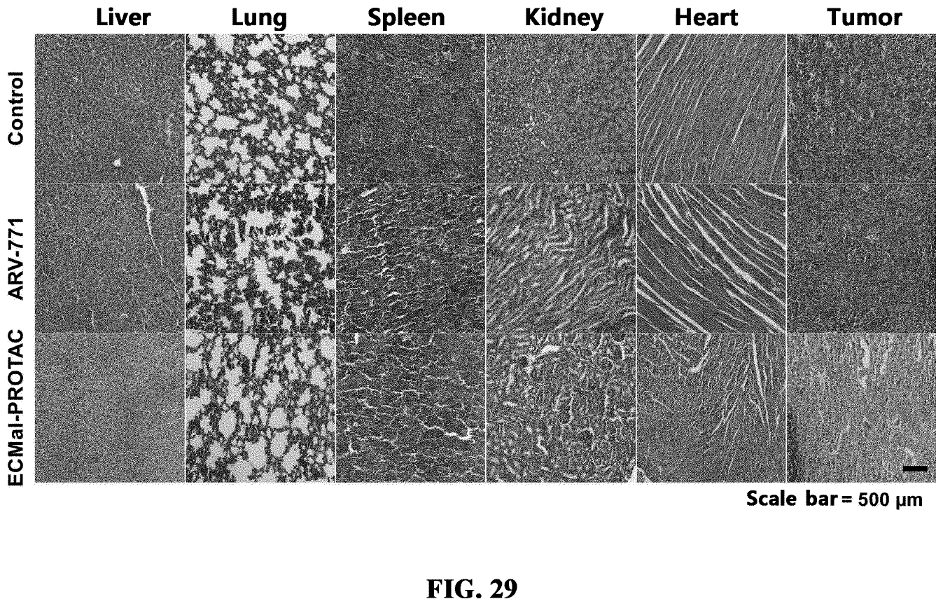

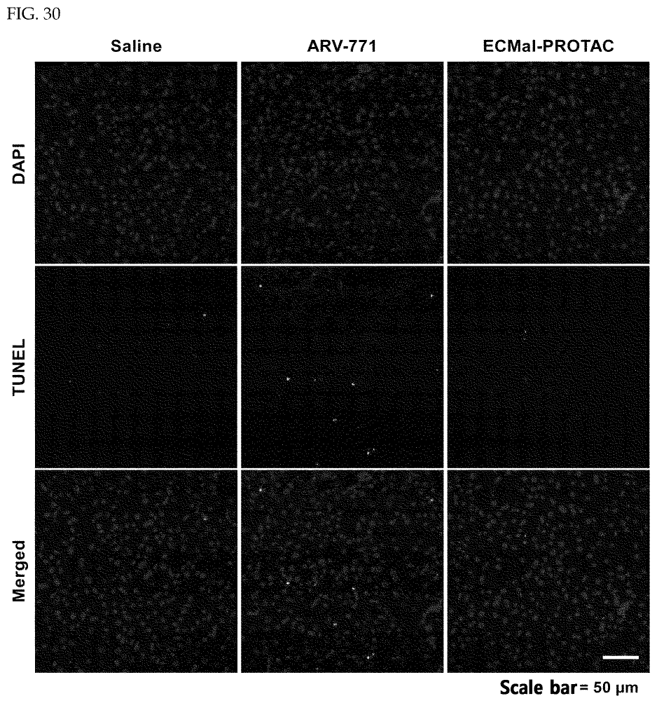

FIG. 29 is the result of histological analysis on organ tissue (liver, lung, spleen, kidney, heart) and cancer tissue excised from the control group, the comparison group, and the example group.

According to FIG. 29, major organ tissue and cancer tissue were excised from the control group, the comparison group, and the example group after 15 days. The excised organ tissue and cancer tissue were stained with H&E (hematoxylin & eosin). The H&E stained tissue was observed using a BX51 optical microscope (Olympus, Tokyo, Japan) and a TCS SP8 confocal laser microscope.

According to FIG. 29, strong damage was confirmed in the cancer tissue of the example group treated with the complex for cancer targeting (ECMal-PROTAC) of example 1. No significant damage was confirmed in the organ tissue excluding the cancer tissue. The comparison group administered with ARV-771 did not observe serious damage in the organ tissue as well as the cancer tissue. That is, administration of ARV-771 does not harm normal tissue, but it was confirmed that it also does not harm cancer tissue (poor anticancer efficacy).

A particularly noteworthy point is that, despite the previous biodistribution example confirming that a significant portion of the complex for cancer targeting (ECMal-PROTAC) of example 1 was delivered to the liver tissue as well as the cancer tissue, the complex for cancer targeting (ECMal-PROTAC) of example 1 does not cause damage to the liver tissue. That is, it can be seen that even if the complex for cancer targeting (ECMal-PROTAC) of example 1 accumulates in normal tissue, it does not cause apoptosis if it is not cancer tissue, and it only causes specific damage and apoptosis to cancer tissue.

FIG. 30 is the result of TUNEL analysis on liver tissue excised from the control group, the comparison group, and the example group.

According to FIG. 30, no significant difference in the apoptosis rate was observed in the liver tissue excised from the example group compared to the liver tissue of the control group. Through these results, it can be seen that when the complex for cancer targeting (ECMal-PROTAC) of example 1 is administered in vivo, a high concentration accumulates in the liver tissue. However, it exhibits almost no toxicity to normal cells, and only exhibits specific activity toward cancer cells.

When conventional ARV-771 was administered alone (comparison group), it was confirmed to affect both normal tissue and cancer tissue.

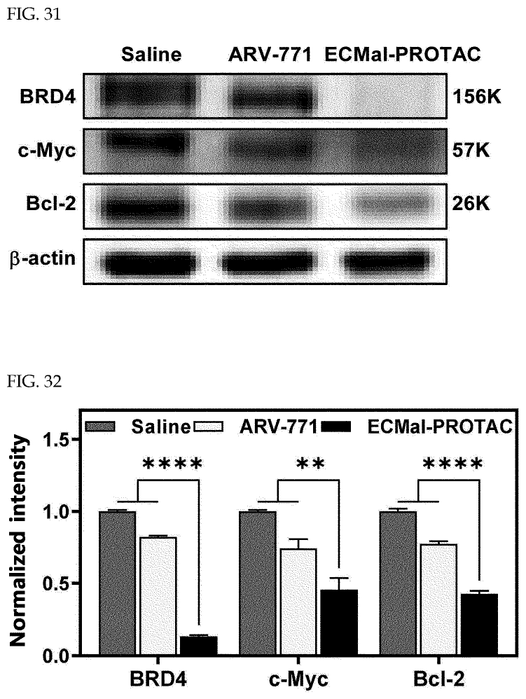

FIG. 31 is the result of Western blot analysis of the expression levels of BRD4, c-Myc, and Bcl-2 in cancer tissue excised from the control group, the comparison group, and the example group. FIG. 32 is a graph quantifying and showing the results of FIG. 31 (**p<0.01, ****p<0.0001).

According to FIG. 31, the cancer tissue of the example group administered with the complex for cancer targeting (ECMal-PROTAC) of example 1 was confirmed to have much lower levels of BRD4, c-Myc, and Bcl-2 than the cancer tissue of the control group. In contrast, no significant difference was observed in the levels of BRD4, c-Myc, and Bcl-2 between the cancer tissue of the example group administered with the complex for cancer targeting (ECMal-PROTAC) of example 1 and the cancer tissue of the comparison group.

According to FIG. 32, the relative expression levels of BRD4, c-Myc, and Bcl-2 in the cancer tissue of the example group administered with the complex for cancer targeting (ECMal-PROTAC) of example 1 were confirmed to be 7.5 times, 2.2 times, and 2.3 times lower, respectively, than the control group.

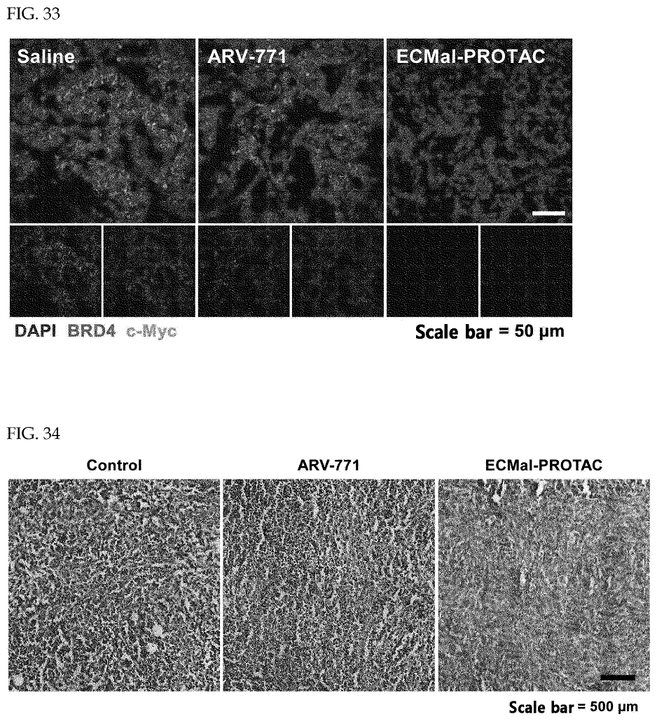

FIG. 33 is an image taken with a confocal laser scanning microscope after staining cancer tissue excised from the control group (Control, Saline), the comparison group (ARV-771, intravenous injection), and the example group (ECMal-PROTAC, intravenous injection) with DAPI, anti-BRD4, and anti-c-Myc.

According to FIG. 33, the example group treated with the complex for cancer targeting (ECMal-PROTAC) of example 1 was confirmed to have weaker fluorescence signals related to c-Myc and BRD4 compared to the comparison group and the control group. In the cancer tissue of the comparison group administered with ARV-771, BRD4 degradation was confirmed to be insufficient to induce apoptosis in cancer tissue. This shows that sufficient anticancer effect did not appear in the comparison group.

FIG. 34 is the result of DAB staining of cancer tissue excised from the control group, the comparison group, and the example group. According to this, BRD4 deficiency was confirmed in the cancer tissue of the example group administered with the complex for cancer targeting (ECMal-PROTAC) of example 1.

That is, the complex for cancer targeting (ECMal-PROTAC) of example 1 was confirmed to successfully degrade and reduce BRD4 in cancer tissue in vivo. The general mechanism of BRD4 deficiency-related cancer cell apoptosis is shown in FIG. 35.

The process of BRD4 deficiency-related cancer cell apoptosis is as follows: c-Myc protein plays an important role in the growth and proliferation of cancer cells, and Bcl-2 induces a cell apoptosis inhibitory effect. Therefore, inducing BRD4 degradation in cancer tissue leads to BRD4 deficiency, and efficiently suppresses the production of c-Myc and Bcl-2 inside the cancer cells, resulting in cell apoptosis. When the complex for cancer targeting (ECMal-PROTAC) of example 1 is administered in vivo, it forms an in situ bond immediately with plasma albumin present in the blood vessel. This results in low systemic toxicity, high specific selectivity toward cancer, and a high absorption rate into cancer cells. Furthermore, it penetrates broadly and deeply even into the central area of the cancer tissue. It suppresses the production of c-Myc and Bcl-2 proteins through the BRD4 degradation pathway inside the cancer cells, inducing cell apoptosis. Thereby, it suppresses the proliferation of cancer cells and causes apoptosis, exhibiting an anticancer effect.

We claim:

1. A complex for cancer targeting, comprising:
(i) a maleimide functional group;
(ii) a linker cleavable by an esterase connected to the functional group; and
(iii) a PROTAC connected to the linker;
wherein the linker is a peptide linker comprising one or more amino acid sequences selected from the group consisting of G, GG, and GGG, a non-peptide linker selected from the group consisting of PABC (p-aminocarbamate), MBA (N,N′-methylenebisacrylamide), alkylene linker, arylene linker, polyalkylene oxide linker (e.g., PEG), NHS ester linker, Triazene linker, hydrazone linker, disulfide linker, glucuronide-MABC linker, Y—S—Z linker, and dialkoxydiphenylsilane linker, or a linker in which the peptide linker and the non-peptide linker are hybridized;
wherein the Y is azide (N₃), alkyl azide having 1 to 6 carbon atoms (—R′—N═N═N), the Z is any one selected from tetrazine, trans-cyclooctene, and cyclooctene, and the S is any one selected from oligo or poly(ethylene glycol) (PEG), dextran, and alkyl having 1 to 10 carbon atoms.

2. The complex for cancer targeting according to claim 1,
wherein the PROTAC may target any one or more proteins selected from the group consisting of CDK9 (Cyclin-dependent kinase 9)/CDK family, BRD4 (Bromodomain-containing protein 4)/BET (Bromodomain and extra-terminal) family, SHP-2 (Src homology region 2 domain-containing phosphatase-2), AR (Androgen receptor), ER (Estrogen receptor), RAR (Retinoic acid receptor), ERRα (Estrogen-related receptor alpha), BTK (Bruton's tyrosine kinase), ALK (Anaplastic lymphoma kinase), RIPK (Receptor-interacting serine/threonine-protein kinase), c-Met (MET proto-oncogene/receptor tyrosine kinase), FAK (Focal adhesion kinase), IRAK4 (Interleukin-1 receptor-associated kinase 4), p38 MAPK (p38 mitogen-activated protein kinases), SGK (Serum and glucocorticoid-regulated kinase), TBK1 (TANK-binding kinase 1), KRAS (Kirsten rat sarcoma virus protein), B-Raf (B-Raf proto-oncogene serine/threonine kinase), β-catenin, FKBP (FK506 binding protein), IDO (Indoleamine 2,3-dioxygenase), PD-1/PD-L1 (Programmed death protein 1/programmed death-ligand 1), PARP1 (Poly [ADP-ribose] polymerase 1), PRC2 (Polycomb repressive complex 2), EGFR (Epidermal growth factor receptor), Sirt2 (NAD-dependent deacetylase sirtuin 2), HER2/ERBB2 (Human epidermal growth factor receptor 2), FRS2 (Fibroblast growth factor receptor substrate 2), BCL-XL (B-cell lymphoma-extra large), SMARCA (SWI/SNF related matrix associated actin dependent regulator of chromatin subfamily A), HDM2 (Human double minute 2 homolog), HDAC (Histone deacetylase) family, BCR-ABL (Breakpoint cluster region protein-Tyrosine-protein kinase ABL1), MCL1 (Modulator Of VRAC Current 1), FLT-3 (Fms Related Receptor Tyrosine Kinase 3), STAT3 (Transcription factor STAT3), Myc family, and BAF complex (Brg/Brahma-associated factors).

3. The complex for cancer targeting according to claim 1,
wherein the linker is Y—S—Z (where Y is an azide (N₃) group or alkyl azide having 1 to 6 carbon atoms (—R′—N═N═N, where R′ is an alkyl group having 1 to 6 carbon atoms), Z is trans-cyclooctene or cyclooctene, and S is poly(ethylene glycol)), or a hybridized linker of a peptide linker consisting of 1 to 3 glycines (G) and a poly(ethylene glycol) non-peptide linker.

4. The complex for cancer targeting according to claim 1,
wherein the complex for cancer targeting is represented by the following Chemical Formula 1 or 2.

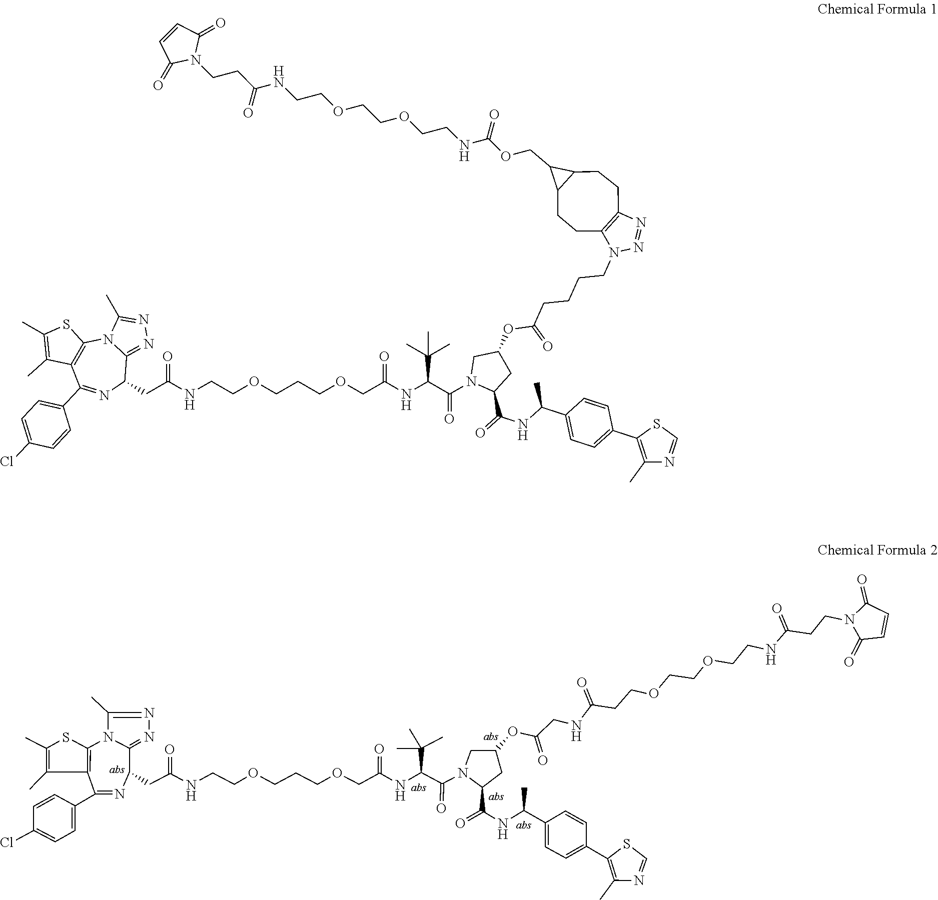

5. The complex for cancer targeting according to claim 1,
wherein the complex for cancer targeting is formed into a stabilized and inactivated form by binding to albumin present in vivo.

6. The complex for cancer targeting according to claim 1,
wherein the complex for cancer targeting penetrates into cancer cells and is cleaved by esterase present in the cancer cells to release PROTAC.

7. A pharmaceutical composition for preventing or treating cancer, comprising the complex for cancer targeting according to claim 1 as an active ingredient.

8. The pharmaceutical composition for preventing or treating cancer according to claim 7,
wherein the complex for cancer targeting is formed into a stabilized and inactivated form by binding to albumin present in vivo.

9. The pharmaceutical composition for preventing or treating cancer according to claim 7,
wherein the cancer is any one or more selected from brain tumor, head and neck cancer, breast cancer, lung cancer, esophageal cancer, stomach cancer, duodenal cancer, appendiceal cancer, colorectal cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, and skin cancer.

10. A food composition for preventing or improving cancer, comprising the complex for cancer targeting according to claim 1 as an active ingredient.
